(19) United States
(12) Patent Application Publication
(10) Pub. No.: US 2018/0360767 A1
(43) Pub. Date: Dec. 20, 2018

(54) BIOCOMPATIBLE PARTICLES AND METHODS OF MAKING AND USE THEREOF

(71) Applicant: NORTH CAROLINA STATE UNIVERSITY, Raleigh, NC (US)

(72) Inventors: Zhen GU, Raleigh, NC (US); Yue LU, Raleigh, NC (US)

(21) Appl. No.: 15/777,349

(22) PCT Filed: Nov. 18, 2016
(86) PCT No.: PCT/US16/62870

§ 371 Date: May 18, 2018

Related U.S. Application Data
(60) Provisional application No. 62/258,287, filed on Nov. 20, 2015.

(51) Int. Cl. A61K 9/51; A61K 31/351; A61K 31/337; A61K 33/24; A61K 47/69; A61K 47/61; A61K 49/04; A61K 49/00; A61P 35/00

(52) U.S. Cl. CPC A61K 9/5161; A61K 9/5115; A61K 31/351; A61K 31/337; A61K 33/24; A61K 47/6923; A61K 9/0019; A61K 47/6951; A61K 49/0428; A61K 49/0093; A61K 49/0032; A61P 35/00; A61K 2123/00; A61K 47/61

(57) ABSTRACT

Biocompatible particles comprising a metallic core and a plurality of drug-loading ligands coordinated to the metallic core are described. The metallic core can, for example, have a melting point of 100° C. or less. The biocompatible particles can, in some examples, further comprise a therapeutically effective amount of a drug coordinated to the plurality of drug-loading ligands. In some examples, the biocompatible particles can further comprise a plurality of targeting ligands coordinated to the metallic core. Also disclosed herein are methods of making the biocompatible particles described herein, for example using sonication. Also disclosed herein are methods of treating cancer in a subject comprising administering to the subject a therapeutically effective amount of the biocompatible particles or compositions disclosed herein.

### CROSS REFERENCE TO RELATED APPLICATIONS

This application claims the benefit of and priority to U.S. Provisional Application No. 62/258,287, filed Nov. 20, 2015, which is incorporated herein by reference in its entirety.

### STATEMENT OF GOVERNMENT SUPPORT

This invention was made with government support under Grant No. 1UL1TR001111 awarded by the National Institute of Health, and Grant Nos. CMMI-0954321 and DMR-1121107 awarded by the National Science Foundation. The government has certain rights in this invention.

### BACKGROUND

The engineering flexibility provided by inorganic nanoparticles with tailorable shape, size, surface ligands, and physical properties has enabled on-demand design of drug delivery systems, contrast agents, and integrated systems for disease diagnosis and treatment. The last decade has witnessed numerous efforts in developing inorganic nanoparticles capable of effectively targeting different diseases. However, these formulations can often fail to be useable due to systemic toxicity. For instance, targeted cancer therapy can require nanoparticles with relatively large sizes to minimize clearance (Soo Choi H et al. Nature Biotech. 2007, 25, 1165-1170) and enhance tumor retention (Tang L et al. Proc. Natl Acad. Sci. USA 2014, 111, 15344-15349; Mitragotri S et al. Nature Rev. Drug Discov. 2014, 13, 655-672), yet such inorganic particles often remain in the body for a long time because of their lack of biodegradability. To date, few studies have demonstrated how to engineer the physicochemical properties of inorganic nanoparticles to satisfy both target delivery and efficient elimination (Choi H S et al. Nature Nanotech. 2010, 5, 42-47; Chou L Y T et al. Nature Nanotech. 2014, 9, 148-155). This design bottleneck is impeding the clinical translation of therapy and diagnostics based on inorganic carriers. The compositions and methods discussed herein address these and other needs.

### SUMMARY

Disclosed herein are biocompatible particles (e.g., a plurality of biocompatible particles). In some examples, the biocompatible particles can comprise a metallic core and a plurality of drug-loading ligands coordinated to the metallic core. The metallic core can, for example, have a melting point of 100° C. or less (e.g., 40° C. or less). In some examples, the metallic core can be liquid at physiological temperatures (e.g., 35° C.-40° C.). In still other examples, the metallic core can have a melting point above 100° C. In some examples, the metallic core can comprise gallium, indium, tin, zinc, or combinations thereof. In some examples, the metallic core can comprise an alloy of gallium and indium, such as a eutectic gallium-indium alloy. In some examples, the metallic core comprises from 95% to 75% gallium and from 25% to 5% indium by weight. The biocompatible particles can have an average particle size of from 5 nm to 2 μm. In some examples, the biocompatible particles can have an average particle size of 200 nm or less.

The plurality of drug-loading ligands can, for example, be selected from the group consisting of natural or synthetic small molecules, macromolecules, nanostructures (e.g., graphene), drug derivatives, and combinations thereof. The drug-loading ligands can be covalently and non-covalently bound to the metallic core. In some examples, the plurality of drug-loading ligands can comprise cyclodextrin, such as thiolated cyclodextrin.

In some examples, the biocompatible particle can further comprise a therapeutically effective amount of a drug coordinated to the plurality of drug-loading ligands. In some examples, the drug can comprise a chemotherapy drug. In some examples, the drug can comprise doxorubicin. In some examples, the drug can comprise an anti-inflammatory drug, anti-infective, a regenerative drug, a biologic (e.g., peptide/protein or nucleic acid), or other drug.

In some examples, the biocompatible particles can further comprise a plurality of targeting ligands coordinated to the metallic core. The plurality of targeting ligands can, for example, comprise peptides, antibodies, proteins, nucleic acids, small molecules, polysaccharides, natural or synthetic polymers, biological membranes, or combinations thereof.

In some examples, the plurality of targeting ligands can comprise a plurality of tumor-targeting ligands. Examples of tumor-targeting ligands include, but are not limited to, antibodies and antibody fragments, and non-antibody ligands like VEGFR or MMP targeting ligands. In some examples, the plurality of targeting ligands comprise hyaluronic acid, such as thiolated hyaluronic acid. Other types of targeting ligands can be used as well, e.g., ligands targeting neuron cells (e.g. dopamine, and other that specifically bind to external motifs of neuronal membrane proteins), inflammatory cells (sugars that can bound to selectins), immune cells (e.g., ligands targeted T-cell receptor), and the like.

In some examples, the biocompatible particles can further comprise a plurality of imaging ligands coordinated to the metallic core. The imaging ligands can be fluorescent, optical, magnetic, radioactive, thermal agents, or combinations thereof.

In some examples, the biocompatible particle can be pH sensitive. Suitable pH sensitive biocompatible particles can be formed from materials that are pH sensitive provided that the resulting biocompatible particles provide for a pH triggered response at the desired pH. Examples of pH triggered responses can include, for example, pH triggered release of the drug, pH triggered degradation of the biocompatible particle, pH triggered increase in X-ray contrast, or combinations thereof. As such, also disclosed herein are: pH-triggered drug-delivery particles comprising the biocompatible particles described herein; pH-triggered biodegradable particles comprising the biocompatible particles described herein; and pH-triggered X-ray contrasts comprising the biocompatible particles described herein. Other types of stimuli-responsive mechanisms can be used, e.g., motifs that respond to physiological and external triggers.

Also disclosed herein are methods of making the biocompatible particles described herein. For example, also disclosed herein are methods of making a biocompatible particle comprising mixing a mixture comprising a metal and a plurality of drug-loading ligands, thereby forming a biocompatible particle comprising a metallic core and a plurality of drug-loading ligands coordinated to the metallic core. Mixing can be accomplished by mechanical stirring, shaking, vortexing, sonication, and the like. In some examples, the mixture can further comprise a plurality of targeting ligands, such that the biocompatible particle further comprises a plurality of targeting ligands coordinated to the metallic core. The method can, in some examples, further comprise loading a therapeutically effective amount of a drug on the plurality of drug-loading ligands.

Also disclosed herein are pharmaceutical compositions comprising the biocompatible particles described herein and a pharmaceutically acceptable excipient.

Also disclosed herein are methods of treating cancer in a subject comprising administering to the subject a therapeutically effective amount of the biocompatible particles or compositions disclosed herein. In some examples, the method of treating cancer in a subject can further comprise co-administering an anticancer agent to the subject.

Also disclosed herein are methods of suppressing tumor growth in a subject, comprising contacting at least a portion of the tumor with a therapeutically effective amount of the biocompatible particles or compositions disclosed herein.

Also disclosed herein are methods of imaging a cell or a population of cells within or about a subject, the method comprising administering to the subject an amount of the biocompatible particles or the compositions disclosed herein; and detecting the biocompatible particles or the compositions disclosed herein. In some examples, the cell or population of cells can be indicative of cancer.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 show a schematic design of the transformable liquid-metal delivery system. Panel a shows the preparation route of doxorubicin loaded ligand-capped liquid metal nanoparticles (LM-NP/Dox-L). Panel b shows the main components of doxorubicin loaded ligand-capped liquid metal nanoparticles: thiolated cyclodextrin (CD) with doxorubicin (Dox), hyaluronic acid (HA) based targeting motif and a gallium-indium eutectic alloy core. Panel c shows the pH-responsive delivery of doxorubicin by doxorubicin loaded ligand-capped liquid metal nanoparticles to nuclei for the targeted cancer therapy: I, accumulation of doxorubicin loaded ligand-capped liquid metal nanoparticles at the tumor site through passive and active targeting; II, specific binding to the overexpressed receptors on the tumor cells; III, receptor-mediated endocytosis; IV, acid-triggered fusion of doxorubicin loaded ligand-capped liquid metal nanoparticles and endosomal/lysosomal escape of doxorubicin containing ligands; V, accumulation of doxorubicin in the nucleus. Panel d shows the acid-triggered fusion and degradation process of doxorubicin loaded ligand-capped liquid metal nanoparticles. Panel e shows the chemical structures of thiolated (2-hydroxypropyl)-β-cyclodextrin (MUA-CD) and thiolated hyaluronic acid (m-HA).

FIG. 2 shows the gallium-indium eutectic alloy was ultrasonically dispersed in ligand containing ethanol mixture (shown schematically in Panel a of FIG. 1).

FIG. 3 is a high-resolution transmission electron microscopy (HRTEM) image of the outer shells of a gallium-indium eutectic alloy particle. Scale bar is 20 nm.

FIG. 4 is an EDS (energy-dispersive X-ray spectroscopy) spectrum of gallium-indium eutectic alloy particles.

FIG. 5 shows the hydrodynamic size of doxorubicin loaded ligand-capped liquid metal nanoparticles measured by dynamic light scattering. Inset: TEM image of doxorubicin loaded ligand-capped liquid metal nanoparticles. Scale bar is 100 nm.

FIG. 6 shows the hydrodynamic size of doxorubicin loaded liquid metal nanoparticles without the hyaluronic acid targeting ligand (LM-NP/Dox) and doxorubicin loaded ligand-capped liquid metal nanoparticles (LM-NP/Dox-L) in 10% fetal bovine serum (FBS).

FIG. 7 shows the release profiles of doxorubicin loaded ligand-capped liquid metal nanoparticles at different pH levels.

FIG. 8 shows the release profiles of LM-NP/PTX-L at different pH levels. Error bars indicate standard deviation (n=3).

FIG. 9 shows the release profiles of doxorubicin loaded gold nanoparticles (GNP/Dox) at different pH levels. Error bars indicate standard deviation (n=3).

FIG. 10 is representative TEM images of doxorubicin loaded ligand-capped liquid metal nanoparticles after different time immersed in acidic (pH 5.0) PBS buffer. Scale bars: 5 min, 1 h and 4 h: 100 nm; 72 h: 400 nm.

FIG. 11 shows the polydispersity index (PDI) of doxorubicin loaded ligand-capped liquid metal nanoparticles immersed in neutral (pH 7.4) and acidic (pH 5.0) PBS buffer.

FIG. 12 shows optical images of the liquid metal nanoparticles before and after acid treatment. Scale bar is 40 km.

FIG. 13 plots the changes of metal ion concentration under neutral and acidic environments.

FIG. 14 shows the light transmittance change over time.

FIG. 15 shows X-ray images of the liquid metal nanoparticles immersed in neutral (pH 7.4) and acidic (pH 5.0) PBS buffer (particles were imaged after 12 h treatment). The images were taken in a 96-well plate. Red lines indicate the areas quantitatively analyzed for grey values (FIG. 16).

FIG. 16 shows the grey value analysis of representative areas in X-ray images of the liquid metal nanoparticles in FIG. 15. Error bars indicated standard deviation (n=3).

FIG. 17 shows the intracellular delivery of doxorubicin loaded ligand-capped liquid metal nanoparticles toward HeLa cells at different time points observed by confocal laser scanning microscopy (CLSM). The cells were incubated with doxorubicin loaded ligand-capped liquid metal nanoparticles at 37° C. for 1 and 4 h, respectively. The late endosomes and lysosomes were stained by LysoTracker Green, and the nuclei were stained by Hoechst 33342. Scale bar is 10 μm.

FIG. 18 shows the relative uptake efficiency of doxorubicin loaded ligand-capped liquid metal nanoparticles on HeLa cells at 4° C. and 37° C. Compared with the cellular uptake of doxorubicin loaded ligand-capped liquid metal nanoparticles at 37° C., the significant decrease in uptake of doxorubicin loaded ligand-capped liquid metal nanoparticles at 4° C. indicated an energy dependent uptake mechanism.

FIG. 19 shows the relative uptake efficiency of doxorubicin loaded ligand-capped liquid metal nanoparticles on HeLa cells in the presence of various endocytosis inhibitors. Error bars indicate standard deviation (n=3). *P<0.05, **P<0.01 compared with the control group (two-tailed Student's t-test). Inhibitor of clathrin-mediated endocytosis: sucrose (SUC) and chlorpromazine (CPZ); inhibitor of caveolin-mediated endocytosis: nystatin (NYS); inhibitor of macropinocytosis: amiloride (AMI); inhibitor of lipid raft: methyl-β-cyclodextrin (MCD). Compared with the cellular uptake of doxorubicin loaded ligand-capped liquid metal nanoparticles without inhibitors as a control, the significant decrease and increase in uptake of doxorubicin loaded ligand-capped liquid metal nanoparticles with inhibitors confirmed the corresponding endocytosis pathways of the nanoparticles.

FIG. 20 shows a representative TEM image of HeLa cells incubated with doxorubicin loaded ligand-capped liquid metal nanoparticles for 1 h. Red arrows show fused nanospheres; green arrows show dispersion of single nanosphere in cytosol. Scale bar: 2 μm.

FIG. 21 shows a representative TEM image of HeLa cells incubated with doxorubicin loaded ligand-capped liquid metal nanoparticles for 4 h. Red arrows show fused nanospheres; green arrows show dispersion of single nanosphere in cytosol. Scale bar: 2 μm.

FIG. 22 shows a representative TEM image of HeLa cells incubated with doxorubicin loaded ligand-capped liquid metal nanoparticles. Red arrows show fused nanospheres after endocytosis; green arrows show single nanospheres before endocytosis. Scale bar: 1 μm. Inset: zoom in of locations of interest, scale bars: 200 nm.

FIG. 23 shows the changes of intracellular metal ion concentrations determined by ICP-MS. Intracellular ion concentrations were normalized by total protein concentration.

FIG. 24 shows representative TEM images of intracellular doxorubicin loaded ligand-capped liquid metal nanoparticles collected from HeLa cells after different incubation time. Scale bars: 100 nm.

FIG. 25 shows the element mapping results of intracellular doxorubicin loaded ligand-capped liquid metal nanoparticles collected from HeLa cells after different incubation time. Scale bars: 5 min, 1 h and 4 h: 100 nm; 24 h: 200 nm.

FIG. 26 shows the changes of surface chemistry of ligand-capped liquid metal nanoparticles (LM-NP/L) during intracellular fusion and degradation monitored by the Time-of-Flight Secondary Ion Mass Spectrometry (TOF-SIMS). Panel a shows the signal intensity distributions of GaO⁻, GaS⁻ and HS⁻ in the ligand-capped liquid metal nanoparticles collected at different incubation time. Each image represents an area of 500 μm×500 μm. MC: number of secondary ions in the brightest pixel. TC: total counts of detected secondary ions. Panel b shows the overlay of the signals of GaO⁻ (red), GaS⁻ (green) and HS⁻ (blue) in the ligand-capped liquid metal nanoparticles collected at different incubation time.

FIG. 27 shows the flow cytometric analysis of HeLa cell apoptosis induced by doxorubicin loaded ligand-capped liquid metal nanoparticles for 12 h using the Annexin V-FITC/DAPI staining.

FIG. 28 shows the HeLa cell apoptosis induced by doxorubicin loaded ligand-capped liquid metal nanoparticles for 20 h using the APO-BrdU TUNEL assay.

FIG. 29 shows the in vitro cytotoxicity of doxorubicin loaded liquid metal nanoparticles without the hyaluronic acid targeting ligand (LM-NP/Dox) and doxorubicin loaded ligand-capped liquid metal nanoparticles (LM-NP/Dox-L) on HeLa cells for 24 h. Error bars indicate standard deviation (n=4). The concentration of ligand-capped liquid metal nanoparticles (LM-NP/L) is equal to the nanocarrier concentration of doxorubicin loaded formulations in each corresponding group; the error bars for doxorubicin loaded ligand-capped liquid metal nanoparticles (LM-NP/Dox-L) at concentration 1.25, 2.5 and 5 mg L⁻¹ are small. *P<0.05, **P <0.01 compared with the doxorubicin solution group (two-tailed Student's t-test).

FIG. 30 shows the in vitro cytotoxicity of doxorubicin loaded ligand-capped liquid metal nanoparticles (LM-NP/Dox-L) on doxorubicin-resistant HeLa cells for 24 h. Error bars indicate standard deviation (n=4). The concentration of ligand-capped liquid metal nanoparticles (LM-NP/L) is equal to the nanocarrier concentration of doxorubicin loaded formulations in each corresponding group. *P<0.05, **P<0.01 compared with the doxorubicin solution group (two-tailed Student's t-test).

FIG. 31 shows the in vivo fluorescence imaging of the HeLa tumor-bearing nude mice at 6, 24, 48 h after intravenous injection of Cy5.5-labelled doxorubicin loaded liquid metal nanoparticles without the hyaluronic acid targeting ligand (Cy5.5-(LM-NP/Dox)) (I) and Cy5.5-labeled doxorubicin loaded ligand-capped liquid metal nanoparticles (Cy5.5-(LM-NP/Dox-L)) (II) at Cy5.5 dose of 30 nmol kg⁻¹. Arrows indicate the sites of tumors.

FIG. 32 shows the ex vivo fluorescence imaging of the tumor and normal tissues harvested from the euthanized HeLa tumor-bearing nude mice at 48 h post injection. The numeric label for each organ is as follows: 1, heart; 2, liver; 3, spleen; 4, lung; 5, kidney; 6, tumor.

FIG. 33 shows the region-of-interest analysis of fluorescent signals from the tumors and normal tissues. Error bars indicated standard deviation (n=3). *P<0.05 (two-tailed Student's t-test).

FIG. 34 shows the time-dependent biodistribution of doxorubicin loaded ligand-capped liquid metal nanoparticles determined by gallium concentration in Hela tumor-bearing nude mice at 1 h, 6 h, 24 h and 48 h after intravenous injection of doxorubicin loaded ligand-capped liquid metal nanoparticles. Error bars indicate standard deviation (n=3).

FIG. 35 shows the time-dependent biodistribution of doxorubicin loaded ligand-capped liquid metal nanoparticles determined by indium concentration in Hela tumor-bearing nude mice at 1 h, 6 h, 24 h and 48 h after intravenous injection of doxorubicin loaded ligand-capped liquid metal nanoparticles. Error bars indicate standard deviation (n=3).

FIG. 36 shows the plasma concentration versus time curves after intravenous injection of the doxorubicin solution and doxorubicin loaded ligand-capped liquid metal nanoparticles (LM-NP/Dox-L) into mice at doxorubicin dose of 2 mg kg⁻¹. Dox/Plasma (% ID mL⁻¹) is the ratio of the doxorubicin amount in the plasma to the total injected dose (ID). Error bars indicate standard deviation (n=3).

FIG. 37 shows the HeLa tumor growth curves after intravenous injection of different formulations of doxorubicin at a dose of 2 mg kg⁻¹. Error bars indicate standard deviation (n=5). *P<0.05, **P<0.01 (two-tailed Student's t-test).

FIG. 38 shows representative images of HeLa xenograft tumors collected from the mice after treatment with different formulations at Day 14. The numeric label for each tumor is as follows: 1, Saline; 2, doxorubicin; 3, doxorubicin loaded liquid metal nanoparticles without the hyaluronic acid targeting ligand; 4, doxorubicin loaded ligand-capped liquid metal nanoparticles. Scale bar is 1 cm.

FIG. 39 shows the body weight variation of HeLa tumor-bearing mice during treatment. Error bars indicate standard deviation (n=5).

FIG. 40 shows representative images of the HeLa xenograft tumors of the mice after treatment with the studied doxorubicin formulations at Day 14. The numeric label for each mouse is as follows: 1, Saline; 2, doxorubicin; 3, doxorubicin loaded liquid metal nanoparticles without the hyaluronic acid targeting ligand; 4, doxorubicin loaded ligand-capped liquid metal nanoparticles. Arrows indicate the sites of tumors.

FIG. 41 shows the histological observation of the tumor tissues after treatment. The tumor sections were stained with hematoxylin and eosin. Scale bar is 100 am.

FIG. 42 shows the histological observation of the normal organs (1. Heart, 2. Liver, 3. Spleen, 4, Lung and 5. Kidney) collected from the mice after treatment with PBS (I) and doxorubicin loaded ligand-capped liquid metal nanoparticles (II) at Day 14. Scale bar is 100 am.

FIG. 43 shows the detection of apoptosis in the tumor tissues after treatment. The tumor sections were stained with fluorescein-dUTP (green) for apoptosis and Hoechst for nuclei (blue). Scale bar is 50 am.

FIG. 44 shows the tumor growth curves after intravenous injection of blank ligand-capped liquid metal nanoparticles (LM-NP/L). Error bars indicate standard deviation (n=5). N.S. (no significance) indicates P>0.05, M-NP/L compared with saline (two-tailed Student's t-test).

FIG. 45 shows the body weight variation of tumor-bearing mice during treatment with blank ligand-capped liquid metal nanoparticles (LM-NP/L). Error bars indicate standard deviation (n=5).

FIG. 46 shows the alanine aminotransferase (ALT), aspartate aminotransferase (AST), alkaline phosphatase (ALP) levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 47 shows the albumin concentration levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 48 shows the blood urea nitrogen (BUN) levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 49 shows the white blood cell levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 50 shows the red blood cell levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 51 shows the hemoglobin levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 52 shows the mean corpuscular volume in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 53 shows the mean corpuscular hemoglobin levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 54 shows the mean corpuscular hemoglobin concentration levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 55 shows the platelet count in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 56 shows the hematocrit levels in the blood of control female Balb/c mice (CK) and female Balb/c mice treated with ligand-capped liquid metal nanoparticles at a dose of 45 mg kg⁻¹ (total nanocarrier dose used in antitumor efficacy study) at 3, 7, 20, 40 and 90 days after ligand-capped liquid metal nanoparticle treatment. Error bars indicate standard deviation (n=3).

FIG. 57 shows the histology evaluation of the major organs (liver, spleen and kidney) collected from the control untreated mice and ligand-capped liquid metal nanoparticle injected mice at different time points post injection. Scale bars: 100 am.

FIG. 58 shows the histology evaluation of organs (heart, brain and muscle) collected from the control untreated mice and ligand-capped liquid metal nanoparticle injected mice (dose: 45 mg kg⁻¹) at day 3 and day 7 post injection. Scale bars: 4 100 am.

FIG. 59 shows the serum IgE levels in female Balb/c mice injected with PBS and ligand-capped liquid metal nanoparticles (dose: 45 mg kg⁻¹). Error bars indicate standard deviation (n=3).

FIG. 60 shows the ligand-capped liquid metal nanoparticle levels in feces and urine determined by gallium in the first week after injection. Error bars indicate standard deviation (n=3).

FIG. 61 shows the ligand-capped liquid metal nanoparticle levels in feces and urine determined by indium in the first week after injection. Error bars indicate standard deviation (n=3).

FIG. 62 shows the release profiles of doxorubicin loaded liquid metal nanoparticles without the hyaluronic acid targeting ligand (LM-NP/Dox) and doxorubicin loaded ligand-capped liquid metal nanoparticles (LM-NP/Dox-L) in 10% fetal bovine serum (FBS).

### DETAILED DESCRIPTION

The biocompatible particles, compositions, and methods described herein may be understood more readily by reference to the following detailed description of specific aspects of the disclosed subject matter and the Examples included therein.

Before the present biocompatible particles, compositions, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “inhibit” refers to a decrease in an activity, response, condition, disease, or other biological parameter. This can include but is not limited to the complete ablation of the activity, response, condition, or disease. This can also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed. For example, the terms “prevent” or “suppress” can refer to a treatment that forestalls or slows the onset of a disease or condition or reduced the severity of the disease or condition. Thus, if a treatment can treat a disease in a subject having symptoms of the disease, it can also prevent or suppress that disease in a subject who has yet to suffer some or all of the symptoms.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

The term “anticancer” refers to the ability to treat or control cellular proliferation and/or tumor growth at any concentration.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

“Half maximal inhibitory concentration” or “IC₅₀”, as used herein, refers to a measure of the effectiveness of a compound in inhibiting biological or biochemical function. This quantitative measure indicates how much of a particular drug or other substance (inhibitor) is needed to inhibit a given biological process (or component of a process, i.e. an enzyme, cell, cell receptor or microorganism) by half. According to the FDA, IC₅₀ represents the concentration of a drug that is required for 50% inhibition in vitro. The IC₅₀ can be determined using a variety of assays known in the art.

Biocompatible Particles

Disclosed herein are biocompatible particles (e.g., a plurality of biocompatible particles). “Biocompatible” and “biologically compatible”, as used herein, generally refer to compounds that are, along with any metabolites or degradation products thereof, generally non-toxic to normal cells and tissues, and which do not cause any significant adverse effects to normal cells and tissues when cells and tissues are incubated (e.g., cultured) in their presence.

In some examples, the biocompatible particles can comprise a metallic core and a plurality of drug-loading ligands coordinated to the metallic core. The size, shape, and composition of the particles (e.g., chemical makeup of the metallic core, the melting point of the metallic core, identity of the ligands coordinated to the metallic core, and combinations thereof) can be selected in view of a variety of factors. The metallic core can, for example, have a melting point of 100° C. or less (e.g., 95° C. or less, 90° C. or less, 85° C. or less, 80° C. or less, 75° C. or less, 70° C. or less, 65° C. or less, 60° C. or less, 55° C. or less, 50° C. or less, 45° C. or less, 40° C. or less, 35° C. or less, 30° C. or less, 25° C. or less, 20° C. or less, 15° C. or less, 10° C. or less, 5° C. or less, 0° C. or less, −5° C. or less, −10° C. or less, −15° C. or less, −20° C. or less, −25° C. or less, −30° C. or less, −35° C. or less, or −40° C. or less). In some examples, the metallic core can have a melting point of 40° C. or less. In some examples, the metallic core can be liquid at physiological temperatures (e.g., 35° C.-40° C.). In certain examples, the metallic core can have a melting point of greater than 100° C. (e.g., 105° C. or more, 110° C. or more, 115° C. or more, 120° C. or more, 130° C. or more, 140° C. or more, or 150° C. or more).

The metallic core can, for example, comprise any biocompatible metal or biocompatible alloy. In some examples, the metallic core can be biocompatible while a metabolite or degradation product of the metallic core (e.g., an ion of the metallic core) can comprise an anticancer agent. In some examples, the metallic core can comprise gallium, indium, tin, zinc, or combinations thereof. As used herein, “combinations thereof” in the context of metals includes alloys. As such, in some examples, the metallic core can comprise an alloy of gallium and indium, such as a eutectic gallium-indium alloy. In another example, galinstan can be used, which is an alloy of gallium, indium, and tin.

In some examples, the metallic core can comprise gallium and indium at a ratio of gallium:indium of 1:99 or more by weight (e.g., 5:95 or more, 10:90 or more, 15:85 or more, 20:80 or more, 25:75 or more, 30:70 or more, 35:65 or more, 40:60 or more, 45:55 or more, 50:50 or more, 55:45 or more, 60:40 or more, 65:35 or more, 70:30 or more, 75:25 or more, 80:20 or more, 85:15 or more, 90:10 or more, or 95:5 or more).

In some examples, the metallic core can comprise gallium and indium at a ratio of gallium:indium of 99:1 or less by weight (e.g., 95:5 or less, 90:10 or less, 85:15 or less, 80:20 or less, 75:25 or less, 70:30 or less, 65:35 or less, 60:40 or less, 55:45 or less, 50:50 or less, 45:55 or less, 40:60 or less, 35:65 or less, 30:70 or less, 25:75 or less, 20:80 or less, 15:85 or less, 10:90 or less, or 5:95 or less).

The weight ratio of gallium:indium in the metallic core can range from any of the minimum values described above to any of the maximum values described above. For example, the metallic core can comprise gallium and indium at a ratio of gallium:indium of from 1:99 to 99:1 by weight (e.g., from 1:99 to 50:50, from 50:50 to 99:1, from 1:99 to 25:75, from 25:75 to 50:50, from 50:50 to 75:25, from 75:25 to 99:1, or from 5:95 to 95:5). In some examples, the metallic core comprises 75% gallium and 25% indium by weight. In some examples, the metallic core comprises 95% gallium and 5% indium by weight.

The biocompatible particles can have an average particle size. “Average particle size,” “mean particle size,” and “median particle size” are used interchangeably herein, and generally refer to the statistical mean particle size of the particles in a population of particles. For a particle with a substantially spherical shape, the diameter of a particle can refer, for example, to the hydrodynamic diameter. As used herein, the hydrodynamic diameter of a particle can refer to the largest linear distance between two points on the surface of the particle. Mean particle size can be measured using methods known in the art, such as evaluation by scanning electron microscopy, transmission electron microscopy, and/or dynamic light scattering.

The biocompatible particles can, for example, have an average particle size of 5 nanometers (nm) or more (e.g., 10 nm or more, 15 nm or more, 20 nm or more, 25 nm or more, 30 nm or more, 35 nm or more, 40 nm or more, 45 nm or more, 50 nm or more, 55 nm or more, 60 nm or more, 65 nm or more, 70 nm or more, 75 nm or more, 80 nm or more, 85 nm or more, 90 nm or more, 95 nm or more, 100 nm or more, 110 nm or more, 120 nm or more, 130 nm or more, 140 nm or more, 150 nm or more, 160 nm or more, 170 nm or more, 180 nm or more, 190 nm or more, 200 nm or more, 225 nm or more, 250 nm or more, 275 nm or more, 300 nm or more, 325 nm or more, 350 nm or more, 375 nm or more, 400 nm or more, 425 nm or more, 450 nm or more, 475 nm or more, 500 nm or more, 550 nm or more, 600 nm or more, 650 nm or more, 700 nm or more, 750 nm or more, 800 nm or more, 850 nm or more, 900 nm or more, 950 nm or more, 1.0 μm or more, 1.1 μm or more, 1.2 μm or more, 1.3 μm or more, 1.4 μm or more, 1.5 μm or more, 1.6 μm or more, 1.7 μm or more, 1.8 μm or more, or 1.9 μm or more).

In some examples, the biocompatible particles can have an average particle size of 2 micrometers (μm) or less (e.g., 1.9 μm or less, 1.8 μm or less, 1.7 μm or less, 1.6 μm or less, 1.5 m or less, 1.4 μm or less, 1.3 μm or less, 1.2 μm or less, 1.1 μm or less, 1.0 μm or less, 950 nm or less, 900 nm or less, 850 nm or less, 800 nm or less, 750 nm or less, 700 nm or less, 650 nm or less, 600 nm or less, 550 nm or less, 500 nm or less, 475 nm or less, 450 nm or less, 425 nm or less, 400 nm or less, 375 nm or less, 350 nm or less, 325 nm or less, 300 nm or less, 275 nm or less, 250 nm or less, 225 nm or less, 200 nm or less, 190 nm or less, 180 nm or less, 170 nm or less, 160 nm or less, 150 nm or less, 140 nm or less, 130 nm or less, 120 nm or less, 110 nm or less, 100 nm or less, 95 nm or less, 90 nm or less, 85 nm or less, 80 nm or less, 75 nm or less, 70 nm or less, 65 nm or less, 60 nm or less, 55 nm or less, 50 nm or less, 45 nm or less, 40 nm or less, 35 nm or less, 30 nm or less, 25 nm or less, 20 nm or less, 15 nm or less, or 10 nm or less).

The average particle size of the biocompatible particles can range from any of the minimum values described above to any of the maximum values described above. For example, the biocompatible particles can have an average particle size of from 5 nm to 2 μm (e.g., from 5 nm to 1 μm, from 1 μm to 2 μm, from 5 nm to 500 nm, from 5 nm to 400 nm, from 5 nm to 300 nm, from 5 nm to 200 nm, from 5 nm to 100 nm, from 100 nm to 200 nm, from 5 nm to 50 nm, from 50 nm to 100 nm, from 100 nm to 150 nm, from 150 nm to 200 nm, from 5 nm to 175 nm, or from 5 nm to 150 nm).

In some examples, the biocompatible particles can be substantially monodisperse. “Monodisperse” and “homogeneous size distribution,” as used herein, and generally describe a population of particles where all of the particles are the same or nearly the same size. As used herein, a monodisperse distribution refers to particle distributions in which 80% of the distribution (e.g., 85% of the distribution, 90% of the distribution, or 95% of the distribution) lies within 25% of the median particle size (e.g., within 20% of the median particle size, within 15% of the median particle size, within 10% of the median particle size, or within 5% of the median particle size).

The biocompatible particles can comprise biocompatible particles of any shape (e.g., a sphere, a rod, a quadrilateral, an ellipse, a triangle, a polygon, etc.). In some examples, the biocompatible particles are substantially spherical.

One or more ligands can be attached to the metallic core, for example, by coordination bonds. Ligands can also be associated with the metallic core via non-covalent interactions. In some examples, the ligands can individually be selected to be a hydrophilic, hydrophobic, or amphiphilic. In addition, the plurality of ligands can, in combination, be selected so as to provide a shell surrounding the metallic core which is hydrophilic, hydrophobic, or amphiphilic. The ligands can comprise metallic coordinating or bonding functional groups, such as thiol, amine, phosphine, CO, N₂, alkene, chloride, hydride, alkyl, and derivatives thereof, and combinations thereof.

The plurality of drug-loading ligands can, for example, be selected from the group consisting of small molecules, macromolecules (synthetic and/or natural), nanostructures (e.g., graphene), drug derivatives (e.g., chemically modified drugs), and combinations thereof. In some examples, the plurality of drug-loading ligands can comprise cyclodextrin, such as thiolated cyclodextrin.

In some examples, the biocompatible particle can further comprise a therapeutically effective amount of a drug coordinated to the plurality of drug-loading ligands. One or more drugs can be attached to the plurality drug-loading ligands, for example, by coordination bonds. Drugs can also be associated with the drug-loading ligands via non-covalent interactions. The identity of the drug-loading ligands can be selected in view of ligand-drug interactions based on a number of factors, including the polarity or charge state of the drug of interest.

A drug refers to a compound or composition that when administered to a subject, will cure, or at least relieve to some extent, one or more symptoms of, a disease or disorder. Drugs include those agents capable of direct toxicity and/or capable of inducing toxicity towards healthy and/or unhealthy cells in the body. Also, the drug can be capable of inducing and/or priming the immune system against potential pathogens.

In some examples, the drug can comprise a chemotherapy drug. Chemotherapy is the treatment of cancer with one or more cytotoxic anti-neoplastic drugs (e.g., chemotherapy drugs) as part of a standardized regimen. Chemotherapy may be given with a curative intent or it may aim to prolong life or to palliate symptoms. In some cases, it can be used in conjunction with other cancer treatments, such as radiation therapy, surgery, hyperthermia therapy, or a combination thereof. Examples of chemotherapy drugs include, for example, 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. In some examples, the drug can comprise doxorubicin. In some examples, the drug can comprise paclitaxel, cisplatin, etc.

In some examples, the drug can comprise an anti-inflammatory drug, an anti-infective, an regenerative drug, a biologic (e.g., a peptide, protein, or nucleic acid).

In some examples, the biocompatible particles can further comprise a plurality of targeting ligands coordinated to the metallic core. The plurality of targeting ligands can, for example, comprise peptides, antibodies, proteins, nucleic acids, small molecules, polymers (natural and synthetic), polysaccharides, biological membranes, or combinations thereof. U.S. Pat. No. 6,960,648 and U.S. Application Publication Nos. 20030032594 and 20020120100 disclose amino acid sequences that can be coupled to another composition and that allows the composition to be translocated across biological membranes. U.S. Application Publication No. 20020035243 also describes compositions for transporting biological moieties across cell membranes for intracellular delivery.

In some examples, the plurality of targeting ligands can comprise a plurality of tumor-targeting ligands. Examples of tumor-targeting ligands include, but are not limited to, antibodies and antibody fragments, and non-antibody ligands like VEGFR or MMP targeting ligands. In some examples, the plurality of targeting ligands comprise hyaluronic acid, such as thiolated hyaluronic acid. Other types of targeting ligands can be used as well, e.g., ligands targeting neuron cells (e.g. dopamine, and other that specifically bind to external motifs of neuronal membrane proteins), inflammatory cells (sugars that can bound to selectins), immune cells (e.g., ligands targeted T-cell receptor), and the like.

In some examples, the biocompatible particle can comprise a plurality of additional ligands, such as a plurality of detectable ligands. The plurality of detectable ligands can comprise, for example, a detectable moiety such as a fluorescence moiety, an optical moiety, a magnetic moiety, a radioactive moiety, a radio-labeled moiety, a thermal moiety, and the like, and combinations thereof.

In some examples, the biocompatible particle can be pH sensitive. Suitable pH sensitive biocompatible particles can be formed from materials that are pH sensitive provided that the resulting biocompatible particles provide for a pH triggered response at the desired pH. The term “pH triggered response” is intended to mean that the response of the biocompatible particle is dependent on or regulated by the pH of the media or environment surrounding the biocompatible particle. For example, suitable pH sensitive biocompatible particles include biocompatible particles that provide for the response at a threshold pH of 6.8 or less (e.g., 6.5 or less, 6 or less, or 5.5 or less). In some cases, the response is triggered by the decrease in endosomal pH initiated by cellular uptake of the biocompatible particles.

Examples of pH triggered responses can include, for example, pH triggered release of the drug, pH triggered degradation of the biocompatible particle, pH triggered increase in X-ray contrast, or combinations thereof. As such, for example, the term “pH triggered release” is intended to mean that the rate of release of the drug from the biocompatible particle is dependent on or regulated by the pH of the media or environment surrounding the biocompatible particle. In some cases, release of the drug (e.g., the chemotherapy drug) is triggered by the decrease in endosomal pH initiated by cellular uptake of the biocompatible particle. The drug is then delivered at the level of the graft. As such, also disclosed herein are pH-triggered drug-delivery particles comprising the biocompatible particles described herein.

In such examples, degradation of the particle can be triggered by the decrease in endosomal pH initiated by cellular uptake of the biocompatible particle. As such, also disclosed herein are pH-triggered biodegradable particles comprising the biocompatible particles described herein. As discussed above, in some examples the metallic core can be biocompatible while a metabolite or degradation product of the metallic core (e.g., an ion of the metallic core) can comprise an anticancer agent. In such examples, degradation of the biocompatible particle can also comprise treatment with an anticancer agent.

In some examples, degradation of the biocompatible particle can lead to an agglomeration of the metallic cores and concomitant increase in average particle size. The increase in average particle size can also lead to an increase in contrast of an image of the particles, such as an X-ray image. As such, also disclosed herein are pH-triggered X-ray contrasts comprising the biocompatible particles described herein.

Methods of Making

Also disclosed herein are methods of making the biocompatible particles described herein. For example, also disclosed herein are methods of making a biocompatible particle comprising sonicating a mixture comprising a metal having a melting point of 100° C. or less and a plurality of drug-loading ligands, thereby forming a biocompatible particle comprising a metallic core and a plurality of drug-loading ligands coordinated to the metallic core, wherein the metallic core has a melting point of 100° C. or less. Instead of sonication, mixing can be done by mechanical stirring, vortexing, shaking and the like. In some examples, the mixture can further comprise a plurality of targeting ligands, such that the biocompatible particle further comprises a plurality of targeting ligands coordinated to the metallic core. The method can, in some examples, further comprise loading a therapeutically effective amount of a drug on the plurality of drug-loading ligands.

Methods of Use

Also provided herein are methods of use of the compounds or compositions described herein. Also provided herein are methods for treating a disease or pathology in a subject in need thereof comprising administering to the subject a therapeutically effective amount of any of the compounds or compositions described herein.

Also provided herein are methods of treating, preventing, or ameliorating cancer in a subject. The methods include administering to a subject a therapeutically effective amount of one or more of the compounds or compositions described herein, or a pharmaceutically acceptable salt thereof. The compounds and compositions described herein or pharmaceutically acceptable salts thereof are useful for treating cancer in humans, e.g., pediatric and geriatric populations, and in animals, e.g., veterinary applications. The disclosed methods can optionally include identifying a patient who is or can be in need of treatment of a cancer. Examples of cancer types treatable by the compounds and compositions described herein include bladder cancer, brain cancer, breast cancer, colorectal cancer, cervical cancer, gastrointestinal cancer, genitourinary cancer, head and neck cancer, lung cancer, ovarian cancer, pancreatic cancer, prostate cancer, renal cancer, skin cancer, and testicular cancer. Further examples include cancer and/or tumors of the anus, bile duct, bone, bone marrow, bowel (including colon and rectum), eye, gall bladder, kidney, mouth, larynx, esophagus, stomach, testis, cervix, mesothelioma, neuroendocrine, penis, skin, spinal cord, thyroid, vagina, vulva, uterus, liver, muscle, blood cells (including lymphocytes and other immune system cells). Further examples of cancers treatable by the compounds and compositions described herein include carcinomas, Karposi's sarcoma, melanoma, mesothelioma, soft tissue sarcoma, pancreatic cancer, lung cancer, leukemia (acute lymphoblastic, acute myeloid, chronic lymphocytic, chronic myeloid, and other), and lymphoma (Hodgkin's and non-Hodgkin's), and multiple myeloma. In some examples, the cancer can be selected from the group consisting of breast cancer, colorectal cancer, and prostate cancer.

The methods of treatment or prevention of cancer described herein can further include treatment with one or more additional agents (e.g., an anti-cancer agent or ionizing radiation).

The one or more additional agents and the compounds and compositions or pharmaceutically acceptable salts thereof as described herein can be administered in any order, including simultaneous administration, as well as temporally spaced order of up to several days apart. The methods can also include more than a single administration of the one or more additional agents and/or the compounds and compositions or pharmaceutically acceptable salts thereof as described herein. The administration of the one or more additional agents and the compounds and compositions or pharmaceutically acceptable salts thereof as described herein can be by the same or different routes. When treating with one or more additional agents, the compounds and compositions or pharmaceutically acceptable salts thereof as described herein can be combined into a pharmaceutical composition that includes the one or more additional agents.

For example, the compounds or compositions or pharmaceutically acceptable salts thereof as described herein can be combined into a pharmaceutical composition with an additional anti-cancer agent, such as 13-cis-Retinoic Acid, 2-Amino-6-Mercaptopurine, 2-CdA, 2-Chlorodeoxyadenosine, 5-fluorouracil, 6-Thioguanine, 6-Mercaptopurine, Accutane, Actinomycin-D, Adriamycin, Adrucil, Agrylin, Ala-Cort, Aldesleukin, Alemtuzumab, Alitretinoin, Alkaban-AQ, Alkeran, All-transretinoic acid, Alpha interferon, Altretamine, Amethopterin, Amifostine, Aminoglutethimide, Anagrelide, Anandron, Anastrozole, Arabinosylcytosine, Aranesp, Aredia, Arimidex, Aromasin, Arsenic trioxide, Asparaginase, ATRA, Avastin, BCG, BCNU, Bevacizumab, Bexarotene, Bicalutamide, BiCNU, Blenoxane, Bleomycin, Bortezomib, Busulfan, Busulfex, C225, Calcium Leucovorin, Campath, Camptosar, Camptothecin-11, Capecitabine, Carac, Carboplatin, Carmustine, Carmustine wafer, Casodex, CCNU, CDDP, CeeNU, Cerubidine, cetuximab, Chlorambucil, Cisplatin, Citrovorum Factor, Cladribine, Cortisone, Cosmegen, CPT-11, Cyclophosphamide, Cytadren, Cytarabine, Cytarabine liposomal, Cytosar-U, Cytoxan, Dacarbazine, Dactinomycin, Darbepoetin alfa, Daunomycin, Daunorubicin, Daunorubicin hydrochloride, Daunorubicin liposomal, DaunoXome, Decadron, Delta-Cortef, Deltasone, Denileukin diftitox, DepoCyt, Dexamethasone, Dexamethasone acetate, Dexamethasone sodium phosphate, Dexasone, Dexrazoxane, DHAD, DIC, Diodex, Docetaxel, Doxil, Doxorubicin, Doxorubicin liposomal, Droxia, DTIC, DTIC-Dome, Duralone, Efudex, Eligard, Ellence, Eloxatin, Elspar, Emcyt, Epirubicin, Epoetin alfa, Erbitux, Erwinia L-asparaginase, Estramustine, Ethyol, Etopophos, Etoposide, Etoposide phosphate, Eulexin, Evista, Exemestane, Fareston, Faslodex, Femara, Filgrastim, Floxuridine, Fludara, Fludarabine, Fluoroplex, Fluorouracil, Fluorouracil (cream), Fluoxymesterone, Flutamide, Folinic Acid, FUDR, Fulvestrant, G-CSF, Gefitinib, Gemcitabine, Gemtuzumab ozogamicin, Gemzar, Gleevec, Lupron, Lupron Depot, Matulane, Maxidex, Mechlorethamine, -Mechlorethamine Hydrochlorine, Medralone, Medrol, Megace, Megestrol, Megestrol Acetate, Melphalan, Mercaptopurine, Mesna, Mesnex, Methotrexate, Methotrexate Sodium, Methylprednisolone, Mylocel, Letrozole, Neosar, Neulasta, Neumega, Neupogen, Nilandron, Nilutamide, Nitrogen Mustard, Novaldex, Novantrone, Octreotide, Octreotide acetate, Oncospar, Oncovin, Ontak, Onxal, Oprevelkin, Orapred, Orasone, Oxaliplatin, Paclitaxel, Pamidronate, Panretin, Paraplatin, Pediapred, PEG Interferon, Pegaspargase, Pegfilgrastim, PEG-INTRON, PEG-L-asparaginase, Phenylalanine Mustard, Platinol, Platinol-AQ, Prednisolone, Prednisone, Prelone, Procarbazine, PROCRIT, Proleukin, Prolifeprospan 20 with Carmustine implant, Purinethol, Raloxifene, Rheumatrex, Rituxan, Rituximab, Roveron-A (interferon alfa-2a), Rubex, Rubidomycin hydrochloride, Sandostatin, Sandostatin LAR, Sargramostim, Solu-Cortef, Solu-Medrol, STI-571, Streptozocin, Tamoxifen, Targretin, Taxol, Taxotere, Temodar, Temozolomide, Teniposide, TESPA, Thalidomide, Thalomid, TheraCys, Thioguanine, Thioguanine Tabloid, Thiophosphoamide, Thioplex, Thiotepa, TICE, Toposar, Topotecan, Toremifene, Trastuzumab, Tretinoin, Trexall, Trisenox, TSPA, VCR, Velban, Velcade, VePesid, Vesanoid, Viadur, Vinblastine, Vinblastine Sulfate, Vincasar Pfs, Vincristine, Vinorelbine, Vinorelbine tartrate, VLB, VP-16, Vumon, Xeloda, Zanosar, Zevalin, Zinecard, Zoladex, Zoledronic acid, Zometa, Gliadel wafer, Glivec, GM-CSF, Goserelin, granulocyte colony stimulating factor, Halotestin, Herceptin, Hexadrol, Hexalen, Hexamethylmelamine, HMM, Hycamtin, Hydrea, Hydrocort Acetate, Hydrocortisone, Hydrocortisone sodium phosphate, Hydrocortisone sodium succinate, Hydrocortone phosphate, Hydroxyurea, Ibritumomab, Ibritumomab Tiuxetan, Idamycin, Idarubicin, Ifex, IFN-alpha, Ifosfamide, IL 2, IL-11, Imatinib mesylate, Imidazole Carboxamide, Interferon alfa, Interferon Alfa-2b (PEG conjugate), Interleukin 2, Interleukin-11, Intron A (interferon alfa-2b), Leucovorin, Leukeran, Leukine, Leuprolide, Leurocristine, Leustatin, Liposomal Ara-C, Liquid Pred, Lomustine, L-PAM, L-Sarcolysin, Meticorten, Mitomycin, Mitomycin-C, Mitoxantrone, M-Prednisol, MTC, MTX, Mustargen, Mustine, Mutamycin, Myleran, Iressa, Irinotecan, Isotretinoin, Kidrolase, Lanacort, L-asparaginase, and LCR. The additional anti-cancer agent can also include biopharmaceuticals such as, for example, antibodies.

Many tumors and cancers have viral genome present in the tumor or cancer cells. For example, Epstein-Barr Virus (EBV) is associated with a number of mammalian malignancies. The compounds disclosed herein can also be used alone or in combination with anticancer or antiviral agents, such as ganciclovir, azidothymidine (AZT), lamivudine (3TC), etc., to treat patients infected with a virus that can cause cellular transformation and/or to treat patients having a tumor or cancer that is associated with the presence of viral genome in the cells. The compounds disclosed herein can also be used in combination with viral based treatments of oncologic disease.

Also described herein are methods of suppressing tumor growth in a subject. The method includes contacting at least a portion of the tumor with a therapeutically effective amount of a compound or composition as described herein, and optionally includes the step of irradiating at least a portion of the tumor with a therapeutically effective amount of ionizing radiation. As used herein, the term ionizing radiation refers to radiation comprising particles or photons that have sufficient energy or can produce sufficient energy via nuclear interactions to produce ionization. An example of ionizing radiation is x-radiation. A therapeutically effective amount of ionizing radiation refers to a dose of ionizing radiation that produces an increase in cell damage or death when administered in combination with the compounds described herein. The ionizing radiation can be delivered according to methods as known in the art, including administering radiolabeled antibodies and radioisotopes.

Also disclosed herein are methods of imaging a cell or a population of cells within or about a subject. The methods can comprise administering to the subject an amount of a compound or a composition as described herein; and detecting the compound or the composition. The detecting can involve methods known in the art, for example, positron emission tomography (PET), single-photon emission computed tomography (SPECT), magnetic resonance imaging (MRI), X-ray, microscopy, computed tomography (CT). In some examples, the compound or composition can further comprise a detectable label, such as a radiolabel, fluorescent label, enzymatic label, and the like. Such imaging methods can be used, for example, for assessing the extent of a disease and/or the target of a therapeutic agent.

The methods and compounds as described herein are useful for both prophylactic and therapeutic treatment. As used herein the term treating or treatment includes prevention; delay in onset; diminution, eradication, or delay in exacerbation of signs or symptoms after onset; and prevention of relapse. For prophylactic use, a therapeutically effective amount of the compounds and compositions or pharmaceutically acceptable salts thereof as described herein are administered to a subject prior to onset (e.g., before obvious signs of the disease or disorder), during early onset (e.g., upon initial signs and symptoms of the disease or disorder), or after an established development of the disease or disorder. Prophylactic administration can occur for several days to years prior to the manifestation of symptoms of a disease or disorder. Therapeutic treatment involves administering to a subject a therapeutically effective amount of the compounds and compositions or pharmaceutically acceptable salts thereof as described herein after the disease or disorder is diagnosed.

Compositions, Formulations and Methods of Administration

In vivo application of the disclosed biocompatible particles, and compositions containing them, can be accomplished by any suitable method and technique presently or prospectively known to those skilled in the art. For example, the disclosed biocompatible particles can be formulated in a physiologically- or pharmaceutically-acceptable form and administered by any suitable route known in the art including, for example, oral, nasal, rectal, topical, and parenteral routes of administration. As used herein, the term parenteral includes subcutaneous, intradermal, intravenous, intramuscular, intraperitoneal, and intrasternal administration, such as by injection.

Administration of the disclosed biocompatible particles or compositions can be a single administration, or at continuous or distinct intervals as can be readily determined by a person skilled in the art.

The biocompatible particles disclosed herein can be formulated according to known methods for preparing pharmaceutically acceptable compositions. Formulations are described in detail in a number of sources which are well known and readily available to those skilled in the art. For example, Remington's Pharmaceutical Science by E. W. Martin (1995) describes formulations that can be used in connection with the disclosed methods. In general, the biocompatible particles disclosed herein can be formulated such that a therapeutically effective amount of the biocompatible particle is combined with a suitable excipient in order to facilitate effective administration of the biocompatible particle. The compositions used can also be in a variety of forms. These include, for example, solid, semi-solid, and liquid dosage forms, such as tablets, pills, powders, liquid solutions or suspension, suppositories, injectable and infusible solutions, and sprays. The preferred form depends on the intended mode of administration and therapeutic application. The compositions also preferably include conventional pharmaceutically-acceptable carriers and diluents which are known to those skilled in the art. Examples of carriers or diluents for use with the biocompatible particles include ethanol, dimethyl sulfoxide, glycerol, alumina, starch, saline, and equivalent carriers and diluents. To provide for the administration of such dosages for the desired therapeutic treatment, compositions disclosed herein can advantageously comprise between 0.1% and 100% by weight of the total of one or more of the subject biocompatible particles based on the weight of the total composition including carrier or diluent.

Formulations suitable for administration include, for example, aqueous sterile injection solutions, which can contain antioxidants, buffers, bacteriostats, and solutes that render the formulation isotonic with the blood of the intended recipient; and aqueous and nonaqueous sterile suspensions, which can include suspending agents and thickening agents. The formulations can be presented in unit-dose or multi-dose containers, for example sealed ampoules and vials, and can be stored in a freeze dried (lyophilized) condition requiring only the condition of the sterile liquid carrier, for example, water for injections, prior to use.

Extemporaneous injection solutions and suspensions can be prepared from sterile powder, granules, tablets, etc. It should be understood that in addition to the excipients particularly mentioned above, the compositions disclosed herein can include other agents conventional in the art having regard to the type of formulation in question.

For the treatment of oncological disorders, the biocompatible particles disclosed herein can be administered to a patient in need of treatment in combination with other antitumor or anticancer substances and/or with radiation and/or photodynamic therapy and/or with surgical treatment to remove a tumor. These other substances or treatments can be given at the same as or at different times from the biocompatible particles disclosed herein. For example, the biocompatible particles disclosed herein can be used in combination with mitotic inhibitors such as taxol or vinblastine, alkylating agents such as cyclophosamide or ifosfamide, antimetabolites such as 5-fluorouracil or hydroxyurea, DNA intercalators such as adriamycin or bleomycin, topoisomerase inhibitors such as etoposide or camptothecin, antiangiogenic agents such as angiostatin, antiestrogens such as tamoxifen, and/or other anti-cancer drugs or antibodies, such as, for example, GLEEVEC (Novartis Pharmaceuticals Corporation) and HERCEPTIN (Genentech, Inc.), respectively, or an immunotherapeutic such as ipilimumab and bortezomib.

In certain examples, biocompatible particles and compositions disclosed herein can be locally administered at one or more anatomical sites, such as sites of unwanted cell growth (such as a tumor site or benign skin growth, e.g., injected or topically applied to the tumor or skin growth), optionally in combination with a pharmaceutically acceptable carrier such as an inert diluent. Biocompatible particles and compositions disclosed herein can be systemically administered, such as intravenously or orally, optionally in combination with a pharmaceutically acceptable carrier such as an inert diluent, or an assimilable edible carrier for oral delivery. They can be enclosed in hard or soft shell gelatin capsules, can be compressed into tablets, or can be incorporated directly with the food of the patient's diet. For oral therapeutic administration, the active biocompatible particle can be combined with one or more excipients and used in the form of ingestible tablets, buccal tablets, troches, capsules, elixirs, suspensions, syrups, wafers, aerosol sprays, and the like.

The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active biocompatible particle, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active biocompatible particle can be incorporated into sustained-release preparations and devices.

Biocompatible particles and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the inclusion of agents that delay absorption, for example, aluminum monostearate and gelatin.

Sterile injectable solutions are prepared by incorporating a biocompatible particle and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, biocompatible particles and agents disclosed herein can be applied in as a liquid or solid. However, it will generally be desirable to administer them topically to the skin as compositions, in combination with a dermatologically acceptable carrier, which can be a solid or a liquid. Biocompatible particles and agents and compositions disclosed herein can be applied topically to a subject's skin to reduce the size (and can include complete removal) of malignant or benign growths, or to treat an infection site. Biocompatible particles and agents disclosed herein can be applied directly to the growth or infection site. Preferably, the biocompatible particles and agents are applied to the growth or infection site in a formulation such as an ointment, cream, lotion, solution, tincture, or the like.

Useful solid carriers include finely divided solids such as talc, clay, microcrystalline cellulose, silica, alumina and the like. Useful liquid carriers include water, alcohols or glycols or water-alcohol/glycol blends, in which the biocompatible particles can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers, for example.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the biocompatible particles and agents and pharmaceutical compositions disclosed herein can be determined by comparing their in vitro activity, and in vivo activity in animal models. Methods for the extrapolation of effective dosages in mice, and other animals, to humans are known to the art.

The dosage ranges for the administration of the compositions are those large enough to produce the desired effect in which the symptoms or disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Also disclosed are pharmaceutical compositions that comprise a biocompatible particle disclosed herein in combination with a pharmaceutically acceptable excipient. Pharmaceutical compositions adapted for oral, topical or parenteral administration, comprising an amount of a biocompatible particle constitute a preferred aspect. The dose administered to a patient, particularly a human, should be sufficient to achieve a therapeutic response in the patient over a reasonable time frame, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

Also disclosed are kits that comprise a biocompatible particle disclosed herein in one or more containers. The disclosed kits can optionally include pharmaceutically acceptable carriers and/or diluents. In one embodiment, a kit includes one or more other components, adjuncts, or adjuvants as described herein. In another embodiment, a kit includes one or more anti-cancer agents, such as those agents described herein. In one embodiment, a kit includes instructions or packaging materials that describe how to administer a biocompatible particle or composition of the kit. Containers of the kit can be of any suitable material, e.g., glass, plastic, metal, etc., and of any suitable size, shape, or configuration. In one embodiment, a biocompatible particle and/or agent disclosed herein is provided in the kit as a solid, such as a tablet, pill, or powder form. In another embodiment, a biocompatible particle and/or agent disclosed herein is provided in the kit as a liquid or solution. In one embodiment, the kit comprises an ampoule or syringe containing a biocompatible particle and/or agent disclosed herein in liquid or solution form.

A number of embodiments of the invention have been described. Nevertheless, it will be understood that various modifications may be made without departing from the spirit and scope of the invention. Accordingly, other embodiments are within the scope of the following claims.

The examples below are intended to further illustrate certain aspects of the systems and methods described herein, and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

All chemicals were purchased from Sigma-Aldrich (St. Louis, Mo., USA) unless otherwise stated. Thiol-polyethylene glycol-amine (HS-PEG-NH₂) with the PEG molecular weight of 1000 were purchased from Sigma-Aldrich and Nanocs Inc. (New York, N.Y., USA). Doxorubicin hydrochloride was purchased from BIOTANG Inc. (Lexington, Mass., USA).

The particle size and Zeta potential were measured by the Zetasizer (Nano ZS, Malvern). The fluorescence intensity was measured by a microplate reader (Infinite M200 PRO, Tecan). Transmission electron microscopy (TEM) images of the liquid metal nanoparticles were obtained on a JEOL 2000FX Scanning Transmission Electron Microscope (STEM). High-resolution transmission electron microscopy (HRTEM) image of oxidized shell was obtained on a JEOL 2010 field emission ultra-high resolution STEM. TEM images of cell sections were obtained on a JEOL 1200EX TEM. Element mapping of single particles was obtained on a TITAN STEM. EDS (energy-dispersive X-ray spectroscopy) spectrum was obtained on a FEI Titan 80-300 probe aberration corrected STEM with SuperX EDS system. X-ray imaging was performed on GE eXplore CT120 micro-CT scanner. Laser scanning confocal microscopy images were obtained with the Carl Zeiss LSM 710 Laser Scanning Microscope.

(2-hydroxypropyl)-β-cyclodextrin (CD, 0.279 g), 11-mercaptoundecanoic acid (MUA, 0.048 g) and 4-Dimethylaminopyridine (DMAP, 0.002 g) were dissolved in 20 mL of dry N,N′-dimethylformamide (DMF), followed by the addition of N,N′-Dicyclohexylcarbodiimide (DCC, 0.041 g). The reaction was allowed to proceed at room temperature overnight. The reaction mixture was first purified by filtration. After rotary evaporation, the resulting thiolated (2-hydroxypropyl)-β-cyclodextrin was characterized by ¹H NMR (Varian Gemini 2300). Thiolated (2-hydroxypropyl)-β-cyclodextrin (MUA-CD): ¹H NMR (DMSO d₆, 300 MHz, δ ppm): 1.01, 1.30, 1.51-1.72, 3.20-3.89, 4.39-5.05, 5.51-6.02.

Sodium hyaluronate (HA, 1.08 g, 3 mg/mL) was dissolved in 45 mL of DI water. HS-PEG-NH₂ was dissolved in 45 mL of DI water. Solutions were combined, EDC and NHS were added as powder, and system pH was adjusted to 5.5±0.3 with NaOH/HCl solutions. After 45 min, reaction pH was increased to and maintained at 7.3±0.3. The reaction was allowed to proceed at room temperature for 4 days. After dialysis against DI water for 48 h, the white powder (thiolated hyaluronic acid, m-HA) was obtained by means of freeze-drying, and characterized by ¹H NMR (Varian Gemini 2300). Thiolated hyaluronic acid (m-HA): ¹H NMR (D₂O, 300 MHz, δ ppm): 1.82, 2.60-2.68, 3.07-3.43, 3.46-3.66.

HeLa cells were obtained from Tissue Culture Facility of UNC Lineberger Comprehensive Cancer Center and cultured in DMEM with 10% (v:v) fetal bovine serum (FBS), 100 U mL-¹ penicillin and 100 μg mL⁻¹ streptomycin in an incubator (Thermo Scientific) at 37° C. under an atmosphere of 5% CO₂ and 90% relative humidity. The cells were sub-cultivated approximately every 3 days at 80% confluence using 0.25% (w:v) trypsin at a split ratio of 1:5.

All animals were treated in accordance with the Guide for Care and Use of Laboratory Animals, approved by the Institutional Animal Care and Use Committee (IACUC) of North Carolina State University. To set up the tumor xenograft model, the female nude mice (6 weeks, J:NU, The Jackson Laboratory) were subcutaneously inoculated in the back with 1×10⁷ HeLa cells. The tumor size was monitored by a vernier caliper and the tumor volume (V) was calculated as V=L×W²/2, where L and Wwere the length and width of the tumor, respectively.

To date, numerous inorganic nanocarriers have been explored for drug delivery systems (DDSs). However, the clinical application of inorganic formulations can be hindered by their toxicity and failure to biodegrade. Herein, a “transformable” liquid-metal nanomedicine, based on a core-shell nanosphere composed of a liquid-phase eutectic alloy of gallium and indium (EGaIn) core and a thiolated polymeric shell, is described. The liquid metal-based nano-scale formulation for drug delivery described herein can, for example, achieve enhanced anticancer therapy.

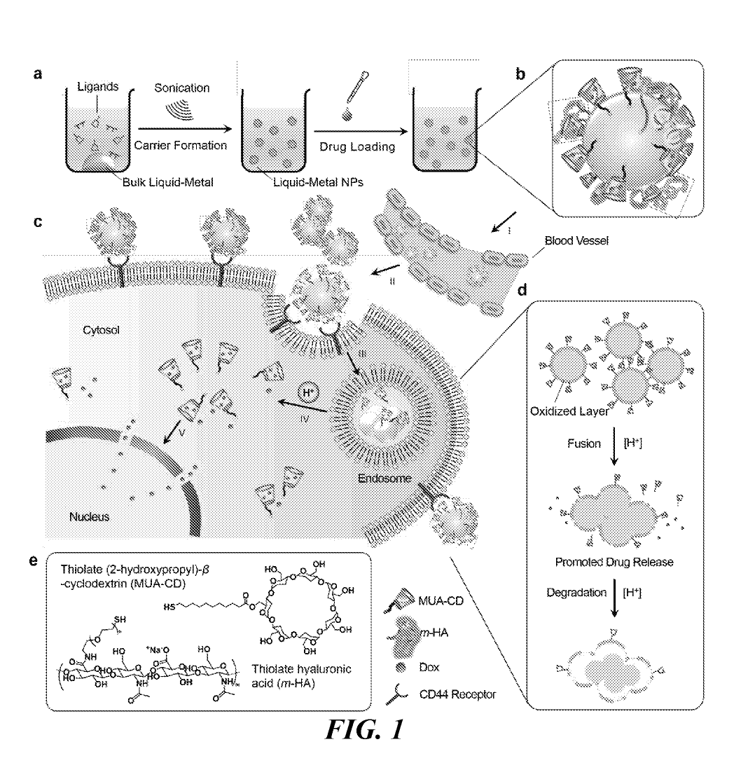

The nanomedicine described herein takes advantage of the certain characteristics of a eutectic alloy of gallium and indium (EGaIn, 75% Ga and 25% In by weight). This nanomedicine can have a variety of merits, such as simple fabrication, facile surface bioconjugation, and the capability of fusion and degradation in a mildly acidic environment. The gallium-indium eutectic alloy is a low-viscosity liquid at room temperature. Unlike Hg, the gallium-indium eutectic alloy has low-toxicity and has thus attracted attention for applications in microfluidic systems, soft robotics and stretchable electronics. To obtain drug nanocarriers based on the gallium-indium eutectic alloy, an “emulsion”-like ligand-mediated procedure was applied through ultrasonication at room temperature (FIG. 1, Panel a). During sonication, the thiolated ligands assemble onto the surface of the gallium-indium eutectic alloy, competing with the oxidation process and facilitating control of the particle size (Boley J W et al. Adv. Mater. 2015, 27, 2270; Hohman J N et al. Nano Letters 2011, 11, 5104-5110; Love J C et al. Chem. Rev. 2005, 105, 1103-1169; Ladd C et al. Adv. Mater. 2013, 25, 5081-5085). The two ligands used here, thiolated (2-hydroxypropyl)-β-cyclodextrin (designated MUA-CD) and thiolated hyaluronic acid (designated m-HA), not only serve as capping agents during the formation of nano-scaled liquid-metal spheres, but can also play roles of drug loading matrix and active targeting moiety, respectively. As shown in FIG. 1, Panel b, the final formulation of ligand-capped liquid metal nanoparticles (designated LM-NP/L) comprises three primary functional constituents: a cyclodextrin-based drug loading motif (FIG. 1, Panel e), the targeting ligand hyaluronic acid and a gallium-indium eutectic alloy core. The 7-membered sugar ring of the cyclodextrin can provide faithful loading sites for doxorubicin (Dox) (Davis M E et al. Nature Rev. Drug Discov. 2004, 3, 1023-1035), a model broad-spectrum chemotherapeutic drug. The hyaluronic acid moiety can supports active tumor-targeting toward the receptors including the CD44 receptor, which is overexpressed on the cell surface of a broad variety of tumors, including human cervical cancer and breast cancer (Aruffo A et al. Cell 1990, 61, 1303-1313). As shown in FIG. 1, Panel c, after intravenous injection, ligand-capped liquid metal nanoparticles can accumulate at tumor sites as a result of passive and active targeting effects (Maeda H et al. J. Control. Release 2000, 65, 271-284). Upon endocytosis, the ligand-capped liquid metal nanoparticles can fuse with each other in the mildly acidic endosome microenvironment (Lu Y et al. J. Control. Release 2014, 194, 1-19), which can lead to the dissociation of the doxorubicin-containing ligands and therefore can promote drug release (FIG. 1, Panel d). The aggregates of fused ligand-capped liquid metal nanoparticles can then degrade due to the oxidative corrosion (FIG. 1, Panel d). Furthermore, the main degradation product, Ga(III), can serve as anticancer agent itself to reverse drug resistance in drug-resistant cancer cells (Collery P et al. Crit. Rev. Oncol. Hemat. 2002, 42, 283-296; Wang F et al. Anticancer Res. 2000, 20, 799-808). Taken together, this liquid metal-based nanomedicine is “transformable”: 1) it can be generated from a bulk material; 2) fusion can promote drug release in an acidic cellular environment and 3) it can be biodegraded to reverse drug resistance and avoid potential systemic toxicity.

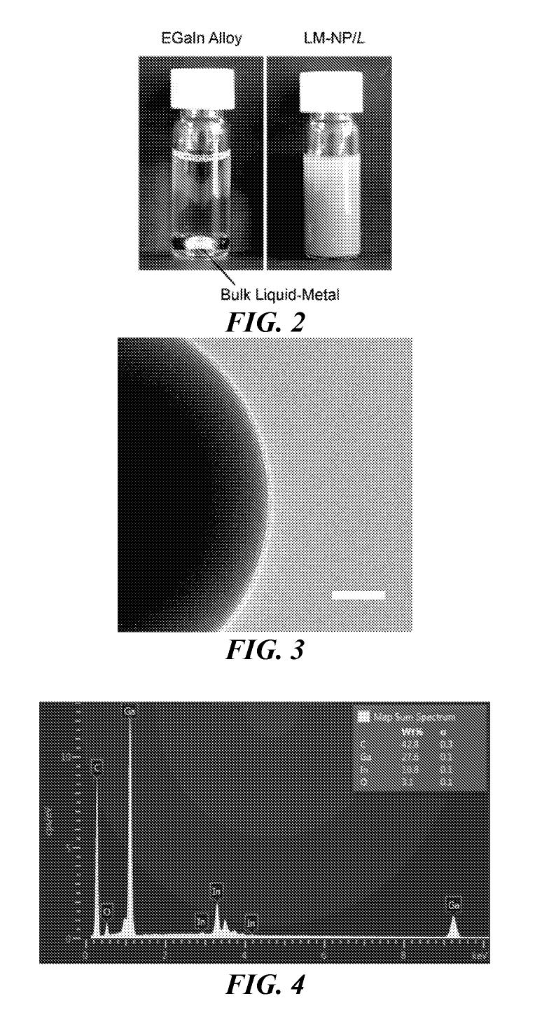

To prepare the ligand-capped liquid metal nanoparticles (LM-NP/L), the gallium-indium eutectic alloy (80 μL) was added to a centrifuge tube (50 mL) filled with an ethanolic solution comprising thiolated cyclodextrin (4 mg) and thiolated hyaluronic acid (0.5 mg) (volume ratio of the gallium-indium eutectic alloy:ethanol=1:150; total volume 12 mL). After sonication, the largest particles precipitated within seconds, and the slurry was removed from the centrifuge tube. The relatively larger particles were separated by mild centrifugation (1000 rpm). The resulting nanoscale colloid comprising nanospheres was removed from the tube and resuspended in water or PB S buffer (FIG. 2).

High-resolution transmission electron microscopy (HRTEM) indicated the particles have a core shell structure (FIG. 3). Energy-dispersive X-ray spectroscopy of the particles indicated that the shell was oxidized (FIG. 4).

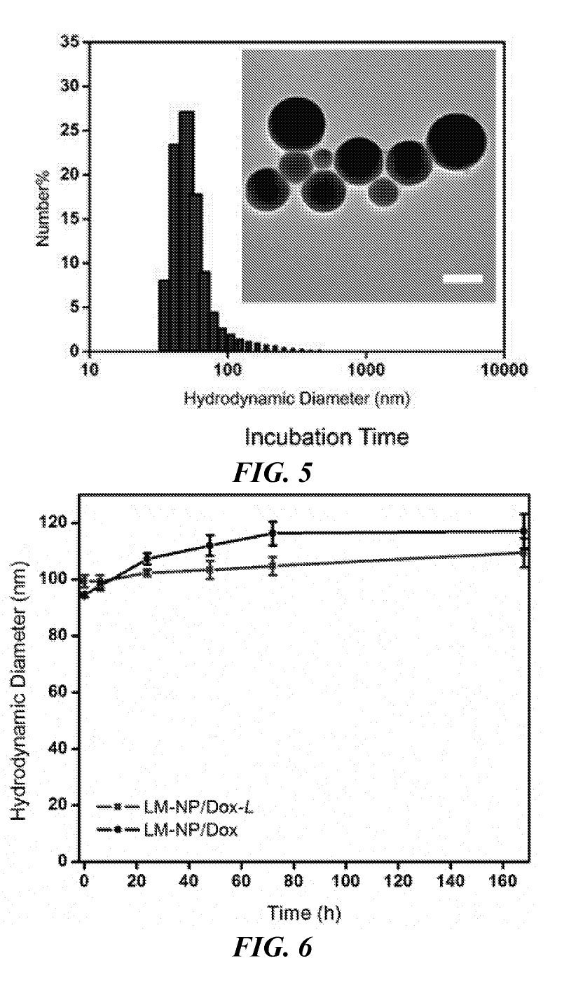

Next, doxorubicin loaded ligand-capped liquid metal nanoparticles (LM-NP/Dox-L) was obtained by overnight incubation, with stirring and at room temperature, of the ligand-capped liquid metal nanoparticles (1 mL) with triethanolamine (TEA)-treated doxorubicin (100 μL) in dimethyl sulfoxide (DMSO) (5 mg mL⁻¹). The mixture was washed (e.g., with PBS via centrifugation) to remove the excess doxorubicin. For characterization by transmission electron microscope (TEM) imaging, the resulting doxorubicin loaded ligand-capped liquid metal nanoparticles were cast onto a TEM copper grid (300 mesh, Ted Pella). After drying in air, the sample was observed by TEM (200 kV, JEM-2000FX, Hitachi). The doxorubicin loaded ligand-capped liquid metal nanoparticles appeared as core-shell structured nanospheres with a diameter of approximately 107 nm in the TEM images, which was consistent with the results determined by dynamic laser scattering (DLS) tests (FIG. 5).

The doxorubicin loading capacity could be tuned by adjusting the ratio between thiolated (2-hydroxypropyl)-β-cyclodextrin and thiolated hyaluronic acid. In a typical formulation used in the following investigations, the doxorubicin loading capacity was determined as 24% (doxorubicin weight ratio of the total weight of nanoparticles). The loading capacity (LC) of doxorubicin loaded ligand-capped liquid metal nanoparticles was calculated as: LC=(A−B)/C, where A was the expected encapsulated amount of doxorubicin, B was the free amount of doxorubicin in the collection solution and C was the total weight of nanoparticles.

For comparison, a sample of liquid metal nanoparticles without the hyaluronic acid targeting ligand (LM-NP) were also synthesized. To prepare the liquid metal nanoparticles without the hyaluronic acid targeting ligand (LM-NP), the gallium-indium eutectic alloy(80 μL) was added to a centrifuge tube (50 mL), which was filled to a total volume of 12 mL with ethanol containing 4 mg thiolated cyclodextrin. After sonication, the largest particles precipitated within seconds, and the slurry was removed from the vial. The particles larger than 100 nm were separated by mild centrifugation (1000 rpm). The resulting nanospheres were collected via centrifugation and resuspended in PBS buffer. Doxorubicin loaded liquid metal nanoparticles without the hyaluronic acid targeting ligand were prepared following the procedure described above.

Comparison of the particle sizes of the ligand-capped liquid metal nanoparticles (LM-NP/L) and the liquid metal nanoparticles without the hyaluronic targeting ligand indicated that the addition of thiolated hyaluronic acid could decrease the average particle size of product (FIG. 6 and Table 1) as well as enhance the systemic stability. These effects can be due to the negative charge of the anionic thiolated hyaluronic acid (Table 1).

TABLE 1
Zeta-potentials and averaged hydrodynamic sizes
of various liquid metal nanoparticles.

| | | Ligand-capped | Doxorubicin loaded |
|---|---|---|---|
| | Liquid metal | liquid metal | ligand-capped liquid |
| | nanoparticles* | nanoparticles | metal nanoparticles |
| | (LM-NP) | (LM-NP/L) | (LM-NP/Dox-L) |
| Zeta-potential | −14.1 ± 0.8 | −24.7 ± 2.1 | −25.1 ± 1.1 |
| (mV) | | | |
| Hydrodynamic | 125 | 104 | 107 |
| size (nm) | | | |

*without the hyaluronic acid targeting ligand

To study the acid-triggered conformational transformation and subsequently promoted drug release, the release profile of doxorubicin from the doxorubicin loaded ligand-capped liquid metal nanoparticles under neutral (1×PBS buffer, pH 7.4) and acidic (1×PBS buffer, pH 5.0) conditions were investigated through the dialysis method (Mo R et al. Nature Commun. 2014, 5, 3364). In short, doxorubicin loaded ligand-capped liquid metal nanoparticles (0.5 mL) were added into a dialysis tube (10 K MWCO) (Slide-A-Lyzer, Thermo Scientific) and dialyzed against 14 mL of a PBS buffer solution at pH 5.0 or 7.4, and gently shaken at 37° C. in a shaker (New Brunswick Scientific) at 100 r.p.m. At predetermined time intervals, the total buffer solution was replaced with 14 mL of fresh buffer solution with the same pH. The fluorescence intensity of doxorubicin released was measured at 596 nm with an excitation wavelength of 480 nm using a microplate reader (Infinite M200 PRO, Tecan).

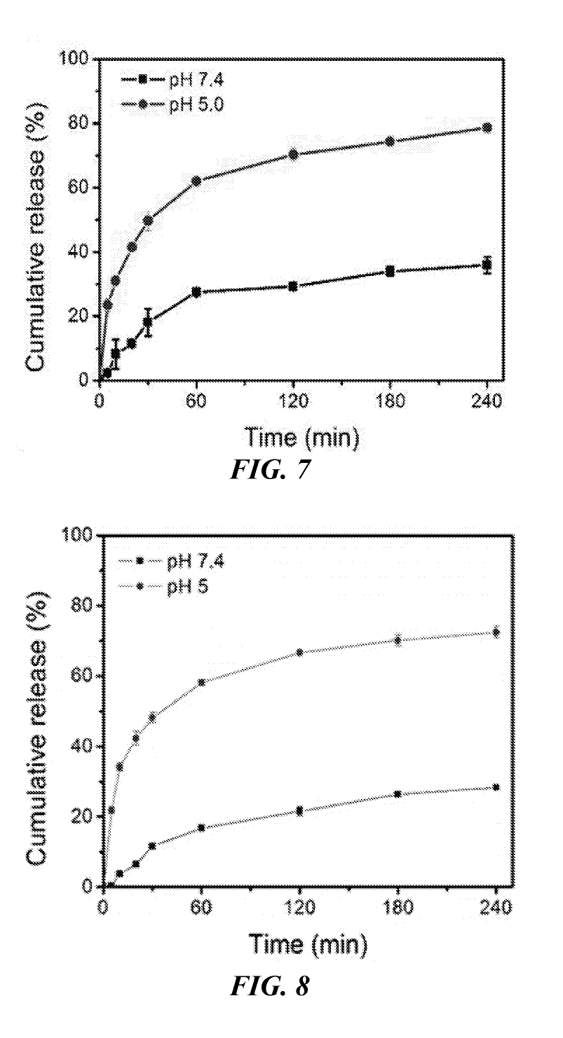

The release profiles of doxorubicin from the doxorubicin loaded ligand-capped liquid metal nanoparticles under neutral (pH 7.4) and acidic (pH 5.0) conditions are shown in (FIG. 7). A burst release of loaded doxorubicin was clearly observed for the doxorubicin loaded ligand-capped liquid metal nanoparticles within the first 30 min in the acidic buffer, which is different from the release curve recorded in the neutral buffer (FIG. 7).

For comparison, Paclitaxel (PTX), which shows insignificant solubility change in the pH range of 2.5 to 8 (Montaseri H. Z. Taxol: Solubility, Stability, and Bioavailability. Doctoral dissertation, University of Alberta, 1997), was loaded onto the ligand-capped liquid metal nanoparticles as a model drug. The paclitaxel loaded ligand-capped liquid metal nanoparticles were synthesized according to the same procedures as for the doxorubicin loaded ligand capped liquid metal nanoparticles. The paclitaxel acid release study was conducted following the same procedure as for the doxorubicin acid release study, except that the concentration of paclitaxel was determined by high-performance liquid chromatography (HPLC, Hewlett Packard 1100). The release profile of the liquid metal nanoparticles loaded with paclitaxel also presented a high release rate within the first 30 min in the acidic buffer (FIG. 8). The acidic buffer caused 72% of the loaded paclitaxel to release within 4 h, which was significantly higher than the 28% in the neutral environment.

Doxorubicin loaded gold nanoparticles (GNP/Dox) with an average diameter of 100 nm, where doxorubicin was included into the thiolated (2-hydroxypropyl)-β-cyclodextrin ligands with same doxorubicin loading capacity as doxorubicin loaded ligand-capped liquid metal nanoparticles, were also applied as a control. To synthesize the doxorubicin loaded gold nanoparticles, gold nanoparticles (diameter: 100 nm, purchased) were first functionalized with mercaptoundecanoic acid (MUA) via ligand exchange reaction. The resulting functionalized gold nanoparticles were then dispersed in dimethylformamide (DMF), followed by the addition of cyclodextrin in presence of dicyclohexylcarbodiimide (DCC) and dimethylaminopyridine (DMAP). The reaction was allowed overnight. After the synthesis, the cyclodextrin functionalized gold nanoparticles (GNPs) were isolated through centrifugation, washed with DI water and filtered using a polyvinylidene fluoride (PVDF) 45 μm filter (Fisherbrand) to remove excess dicyclohexylcarbodiimide. For loading of doxorubicin, 1 mL of gold nanoparticles was incubated with 100 μL of triethanolamine (TEA)-treated doxorubicin in dimethyl sulfoxide (DMSO) (5 mg mL⁻¹) with stirring at room temperature overnight. The resulting doxorubicin-loaded gold nanoparticles (GNP/Dox) were extensively washed with PBS via centrifugation to remove the excess doxorubicin. The surface functionalization degree was fine tuned to achieve the same loading capacity as in the doxorubicin loaded ligand-capped liquid metal nanoparticles (24%).

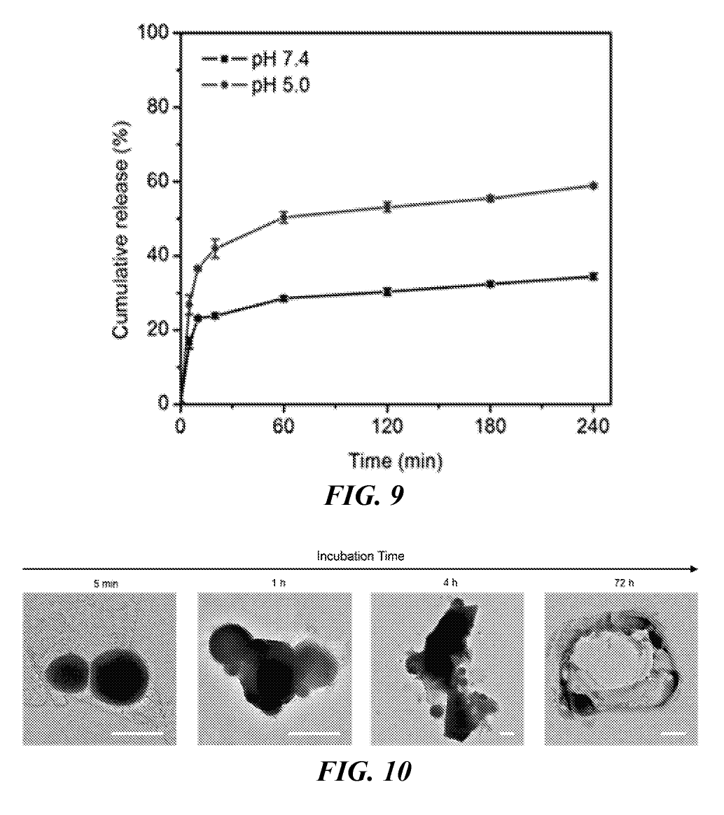

The acid-triggered release profile of doxorubicin from the doxorubicin loaded gold nanoparticles under neutral (pH 7.4) and acidic (pH 5.0) conditions were investigated through the dialysis method described above, the results of which are shown in FIG. 9. Compared with doxorubicin loaded gold nanoparticles (GNP/Dox), the doxorubicin loaded ligand-capped liquid metal nanoparticles (LM-NP/Dox-L) displayed a more potent acid-promoted doxorubicin release at pH 5.0 (FIG. 9 and FIG. 5). This phenomenon can be attributed to fusion of the liquid metal nanoparticles triggered by the disruption of the oxidized shell of the ligand-capped liquid metal nanoparticles, and the subsequent dissociation of surface ligands. There was no significant different in drug release profiles between the doxorubicin loaded liquid metal nanoparticles without the hyaluronic acid targeting ligand (LM-NP/Dox) and the doxorubicin loaded ligand-capped liquid metal nanoparticles (LM-NP/Dox-L) (confirmed by two-tailed Student's t-test, FIG. 62).

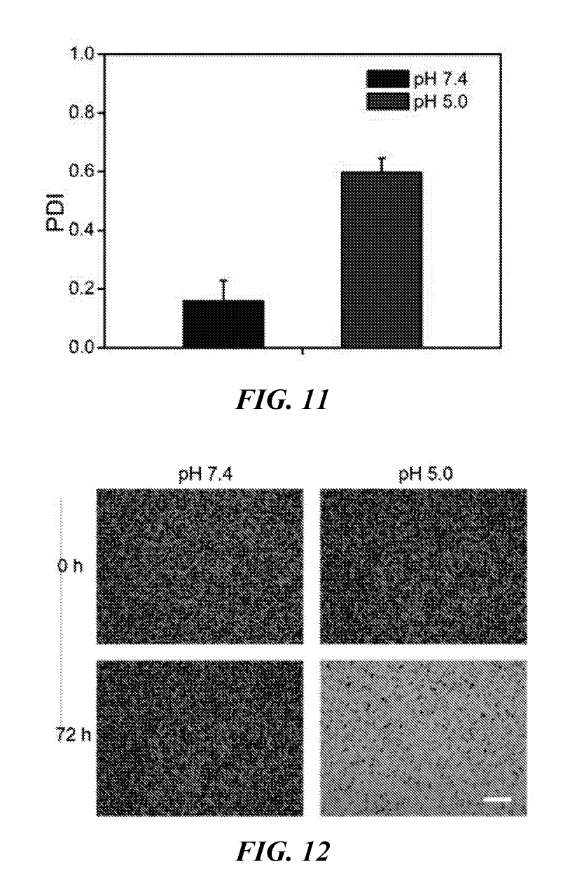

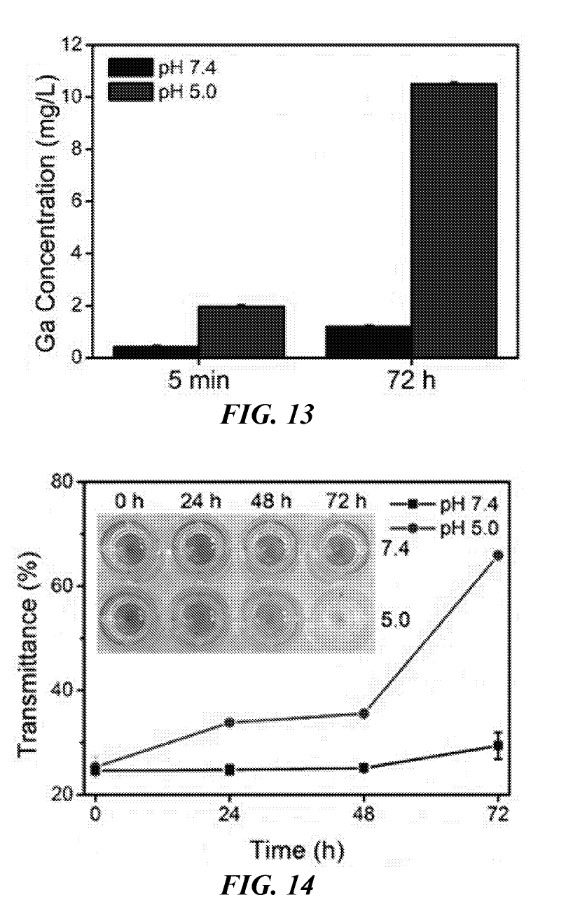

The conformational change of the doxorubicin loaded ligand-capped liquid metal nanoparticles with up to 4 h acid treatment (pH 5.0) was further visualized using transmission electron microscopy (TEM) (FIG. 10). The initial fusion behavior was captured within the first 5 min after acid treatment, and the aggregates formed by fusion increased in size over time. These results were in good agreement with the increase in size distribution (e.g., increase in polydispersity index (PDI)) upon acid treatment as measured by dynamic laser scattering (DLS) (FIG. 11). It was hypothesized that the acid can attack the oxidized layer (Hohman J N et al. Nano Letters 2011, 11, 5104-5110) on the surface of the ligand-capped liquid metal nanoparticles to trigger fusion of the ligand-capped liquid metal nanoparticles, which can further lead to the fusion of the liquid inner cores (Thelen J et al. Lab on a Chip 2012, 12, 3961-3967). The degradation of the doxorubicin loaded ligand-capped liquid metal nanoparticles at pH 5.0 was clearly observed via the optical microscope (FIG. 12) and TEM imaging (FIG. 10) over time; while negligible difference was observed in the case of neutral buffer. The representative TEM image of doxorubicin loaded ligand-capped liquid metal nanoparticles immersed in acidic (pH 5.0) PBS buffer for 72 h showed evidence of degradation into the hollow polymeric shells dotted with shrunk metallic cores in the inner wall evidenced the occurrence of degradation (FIG. 10). To further verify the degradation of the liquid metal nanoparticles, the change of Ga(III) ion concentration in PBS buffer over time was monitored by the inductively coupled plasma optical emission spectrometry (ICP-OES) (FIG. 13). The Ga(III) concentration in the acidic buffer displayed a notable increase over time; while the degradation of the liquid metal nanoparticles in neutral solution was moderate (FIG. 13). The degradation process of the liquid metal nanoparticles was then tracked and quantified by monitoring the change of light transmittance over time (FIG. 14). The light transmittance of the acidic liquid metal nanoparticle solution increased from 25% to 66% (FIG. 14), which was in good agreement of the proposed degradation of the liquid metal nanoparticles in acidic environment. The main degradation product, Ga(III) ion, has been reported to reverse drug resistance in drug-resistant cancer cells (Collery P et al. Crit. Rev. Oncol. Hemat. 2002, 42, 283-296; Wang F et al. Anticancer Res. 2000, 20, 799-808), which could potentially generate synergistic effects and prevent drug resistance.

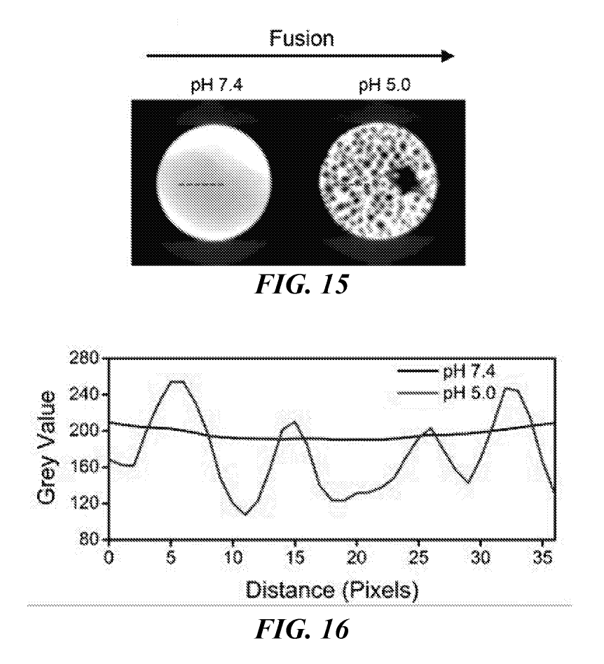

Furthermore, the nature of the gallium-indium eutectic alloy metal core and the verified acid-triggered fusion might also open the possibility of utilizing these transformable nanospheres for imaging contrast enhancement. After acid treatment (12 h in 1 xPBS buffer, pH 5.0), the homogeneous contrast enhancement caused by the liquid metal nanoparticles was disrupted, replaced by heterogeneous contrast within the imaged area with higher contrast at the locations of fused particles and lower contrast at spots in absence of the liquid metal nanoparticles (FIG. 15). The resulting changes in contrast were quantified by studying the grey value profiles of representative areas in X-ray images (FIG. 16). The grey values of the imaging area were approximately constant before acid treatment, indicating homogeneous imaging contrast (FIG. 16). The disruptions of the homogeneous imaging contrast after acid treatment were supported by the appearance of peaks in the grey value plot (FIG. 16). Similar fusion of the liquid metal nanoparticles accumulated at tumor site would potentially lead to enhanced contrast of tumor tissue during X-ray imaging.

The intracellular delivery of the doxorubicin loaded ligand-capped liquid metal nanoparticles into HeLa cells was further explored using the confocal laser scanning microscopy (CLSM). HeLa cells (1×10⁵ cells per well) were seeded in a confocal microscopy dish (MatTek). After culture for 24 h, the cells were incubated with doxorubicin loaded ligand-capped liquid metal nanoparticles (2 μM doxorubicin concentration) at 37° C. for 2 h, and then washed twice using 4° C. PBS, followed by incubation with fresh FBS-free culture medium for additional 0 h or 4 h. Subsequently, the cells were stained by LyosTracker Green (50 nM) (Life Technologies) at 37° C. for 30 min and Hoechst 33342 (1 μg mL⁻¹) (Life Technologies) at 37° C. for 10 min. Finally, the cells were washed by 4° C. PBS twice and immediately observed using confocal laser scanning microscopy (CLSM) (LSM 710, Zeiss).

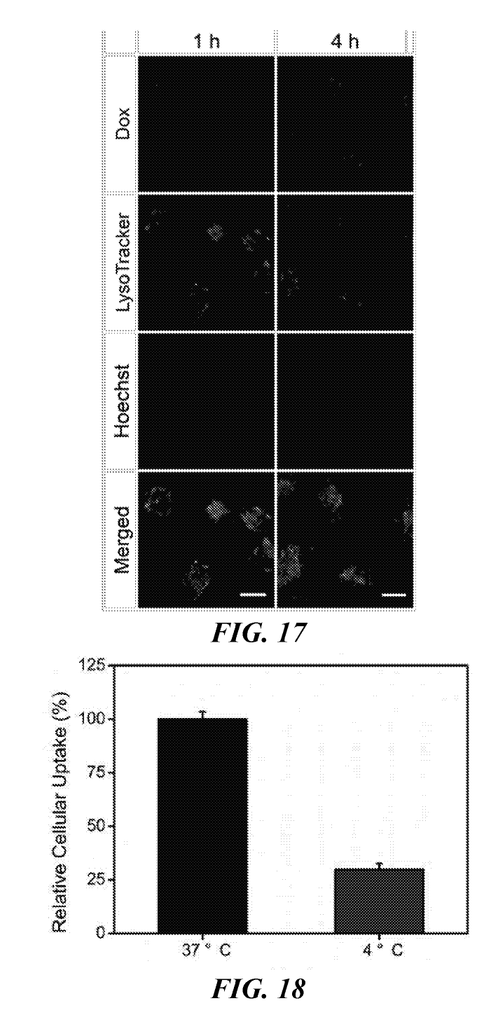

The fluorescence of doxorubicin was clearly observed in HeLa cells after 1 h of incubation with doxorubicin loaded ligand-capped liquid metal nanoparticles, indicating the cellular internalization of the nanospheres (FIG. 17). When the incubation time was prolonged to 4 h, doxorubicin was highly localized within the nuclei of HeLa cells, as indicated by the magenta fluorescence (FIG. 17).

In order to investigate whether the cellular uptake of doxorubicin loaded ligand-capped liquid metal nanoparticles was energy dependent, HeLa cells (1×10⁵ per well) were seeded in the 6-well plates and cultured for 48 h, after which the cells were incubated with doxorubicin loaded ligand-capped liquid metal nanoparticles at a doxorubicin concentration of 2 μM at 4° C. and 37° C., respectively. The cellular uptake of doxorubicin loaded ligand-capped liquid metal nanoparticles displayed an energy-dependent manner (FIG. 18).

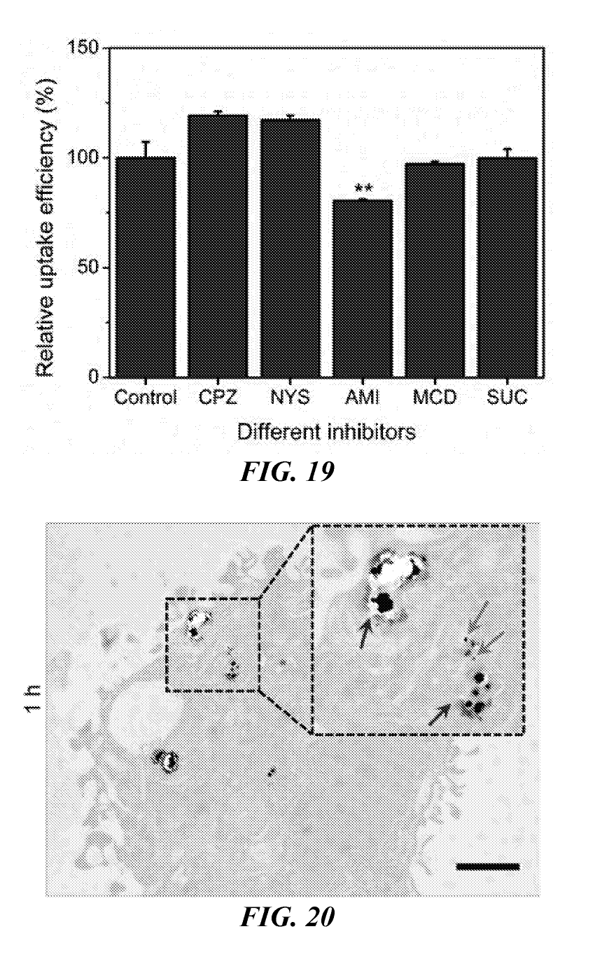

To determine the endocytosis pathway, HeLa cells (1×10⁵ per well) were seeded in the 6-well plates and cultured for 48 h, after which the cells were pre-incubated with several inhibitors for various kinds of endocytosis at 37° C. Sucrose (SUC, 450 mM) and chlorpromazine (CPZ, 10 μM) were used as an inhibitors of clathrin-mediated endocytosis (Hsieh C et al. J. Neurochem. 1999, 73, 493-501; Mo R et al. Nature Commun. 2014, 5, 3364). Nystatin (NYS, 25 μg mL⁻¹) was used as an inhibitor of caveolin-mediated endocytosis (Singh R D et al. Mol. Biol. Cell 2003, 14, 3254-3265). Amiloride (AMI, 1 mM) was used as an inhibitor of micropinocytosis (Sallusto F et al. J. Exp. Med. 1995, 182, 389-400). Methyl-β-cyclodextrin (MCD, 3 mM) was used as an inhibitor of inhibitor of lipid raft endocytosis (Zidovetzki R et al. Biochim. Biophys. Acta 2007, 1768, 1311-1324). Afterward, the cells were incubated with doxorubicin loaded ligand-capped liquid metal nanoparticles at a doxorubicin concentration of 2 μM in the presence of the inhibitors for additional 2 h. After washing the cells with 4° C. PBS twice, the fluorescence intensity of doxorubicin in the cells and the cell proteins were measured, respectively. The cellular uptake of doxorubicin loaded ligand-capped liquid metal nanoparticles indicated that that the doxorubicin loaded ligand-capped liquid metal nanoparticles mainly entered HeLa cells via macropinocytosis (FIG. 19).

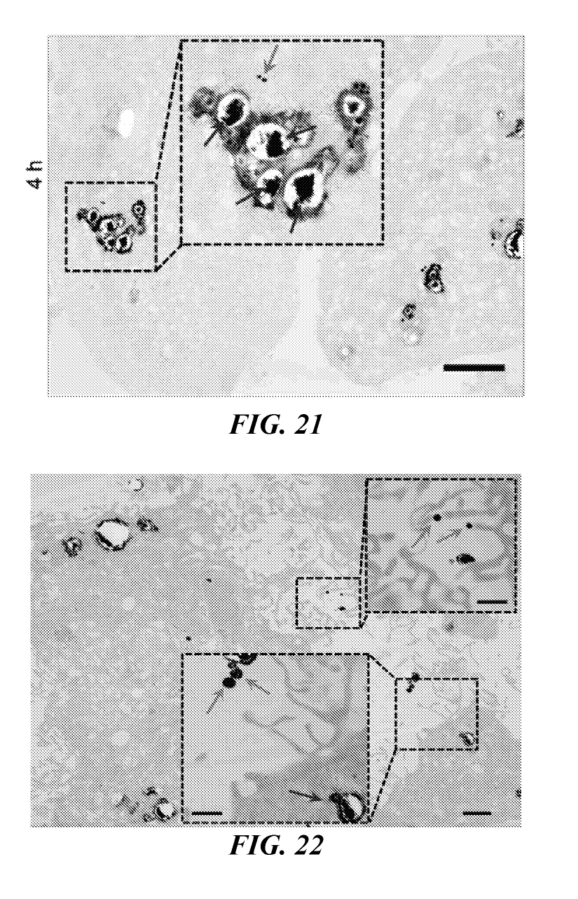

The endocytosis process of doxorubicin loaded ligand-capped liquid metal nanoparticles was also visualized by TEM imaging (FIGS. 20 through 22). HeLa cells were treated with doxorubicin loaded ligand-capped liquid metal nanoparticles for different time durations. After 1 h incubation, fusion of the liquid metal nanoparticles was clearly observed in the acidic endosomes (FIG. 20), agreeing with the in vitro results discussed above. After 4 h incubation, more fusion events of the liquid metal nanoparticles were spotted, together with few single liquid metal nanoparticles dispersed in the cytosol (FIG. 21).

The fusion process was further quantified and visualized intracellularly. HeLa cells (1×10⁵ per well) were seeded in the 6-well plates and cultured for 48 h, followed by incubation with doxorubicin loaded ligand-capped liquid metal nanoparticles. At different time points, the cells were thoroughly washed and lysed. After centrifugation, the supernatants were analyzed for metal ion concentration and total protein concentration. The pellets from centrifugation were carefully washed with DI water and collected for TEM imaging and element analysis.

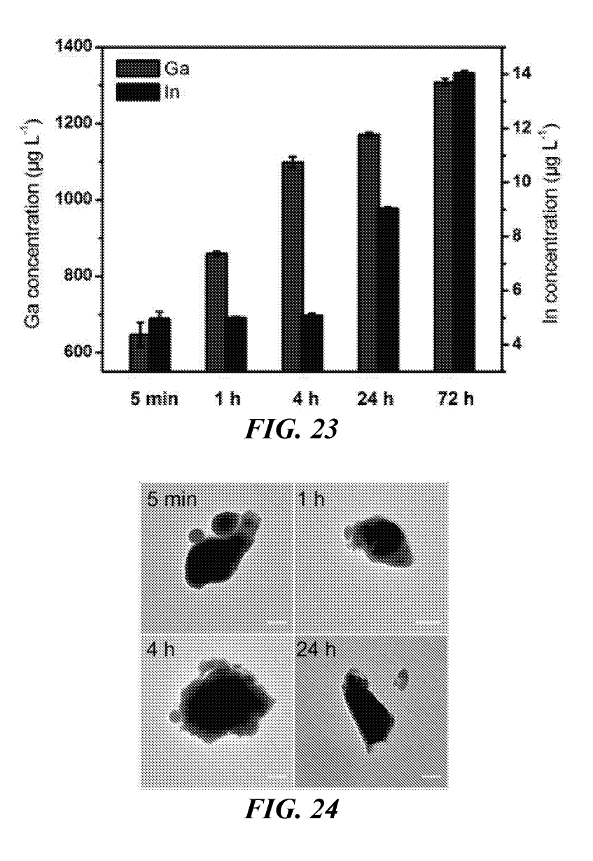

The intracellular Ga/In ion concentrations were then quantified by inductively coupled plasma mass spectrometry (ICP-MS). The intracellular metal ion concentrations were normalized by the total protein concentration (as determined by BCA assay) in the supernatant. The intracellular Ga(III) ion and In ion concentrations both increased over incubation time (FIG. 23).

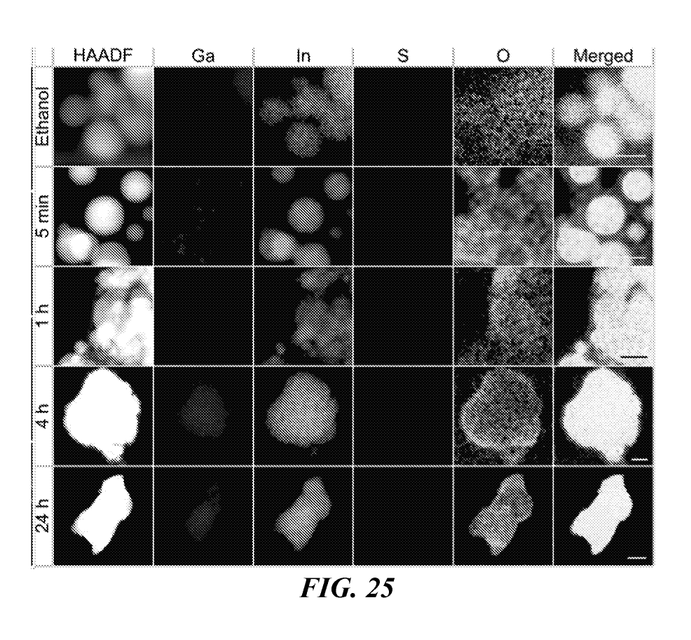

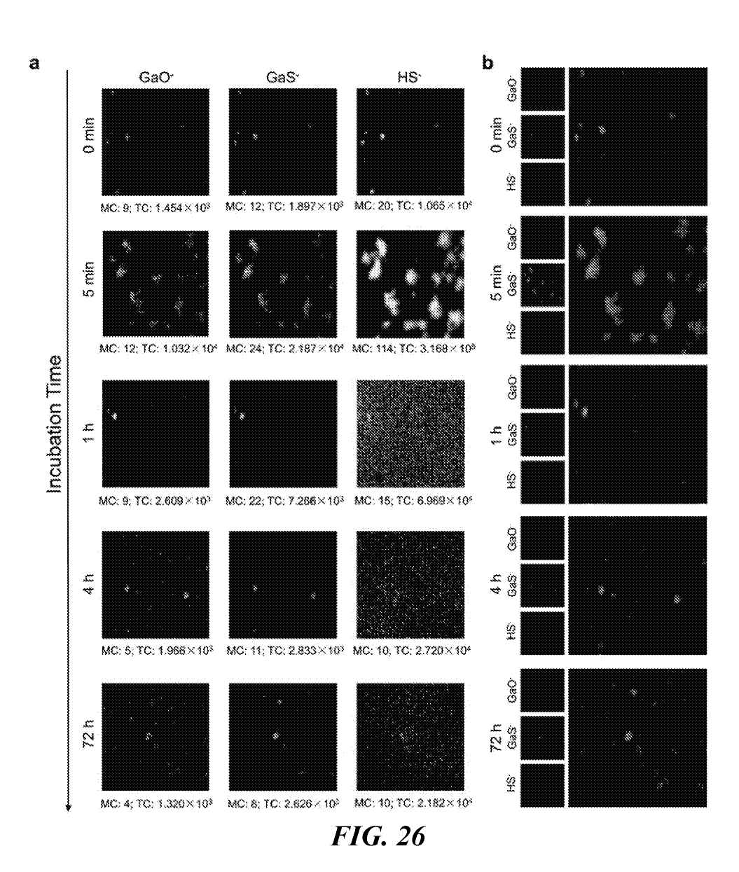

The particle structures formed intracellularly via fusion were also further validated using TEM and analyzed with the Titan scanning/transmission electron microscope (S/TEM) at an atomic scale. Similar to the doxorubicin loaded ligand-capped liquid metal nanoparticles treated with acid (FIG. 10), the initial fusion behavior was captured immediately upon cellular uptake, followed by the formation of larger aggregates and the eventual degradation, (FIG. 24). These results agree with the increase of intracellular Ga(III) ion concentration over incubation time (FIG. 23). The element mapping results revealed the fusion of the polymeric shells during the conformational changes of the ligand-capped liquid metal nanoparticles (FIG. 25). The scattering of Ga signal after 5 min incubation indicated the fast disruption of the oxidized shell. After 1 h of incubation, the increase of S and O signals in the background suggested the dissociation of thiolated surface ligands. The Time-of-Flight Secondary Ion Mass Spectrometry (TOF-SIMS) was further utilized to analyze the surface of the ligand-capped liquid metal nanoparticles and the intracellular ligand-capped liquid metal nanoparticles collected by lysing the cells internalized with particles (FIG. 26). The coexistence of thiols and oxides was well evidenced by the overlapping of the signals of GaO⁻ and GaS⁻ in the ligand-capped liquid metal nanoparticles. The increase of HS signal in the background after 5 min incubation visualized the dissociation of thiolated surface ligands, indicating the initial stage of fusion behavior. The increase in the background HS⁻ signal and the decreases in the signals of GaO⁻ and GaS⁻ indicated that the intracellular fusion behavior of the ligand-capped liquid metal nanoparticles was a result of the synergistic action of the dissociation of thiolated surface ligands and the disruption of oxides.

The Annexin V-FITC/DAPI apoptosis detection and the terminal deoxynucleotidyl transferase dUTP nick end labelling (TUNEL) assay were performed to compare the apoptosis-inducing capabilities of doxorubicin loaded ligand-capped liquid metal nanoparticles and free doxorubicin solution. Annexin V-FITC labels the phosphatidylserine translocated to the extracellular membrane upon the initiation of apoptosis (Vermes I et al. Journal of Immunological Methods 1995, 184, 39-51), while DAPI has significantly higher staining efficiency with cells where the plasma membrane integrity has been compromised (Hakem R et al. Cell 1998, 94, 339-352). This combination allows the differentiation among the early apoptotic cells, the late apoptotic cells and the viable cells, which can be quantitatively determined by the flow cytometry.

Apoptosis of HeLa cells was detected using the APO-BrdU TUNEL Assay Kit (Life Technologies) and Annexin V-FITC Apoptosis Detection Kit (BD Biosciences). The cells (1×10⁵ cells per well) were seeded in the six-well plates. After culture for 48 h, the cells were incubated with doxorubicin-loaded liquid metal nanoparticles for 12 h (Annexin V-FITC) or 20 h (TUNEL). The subsequent procedures were performed in accordance with the manufacturer's protocol. For Annexin V-FITC apoptosis detection, the cells were analyzed by flow cytometry (BD FACSCalibur), while for the TUNEL assay the cells were observed by fluorescence microscope (IX71, Olympus).

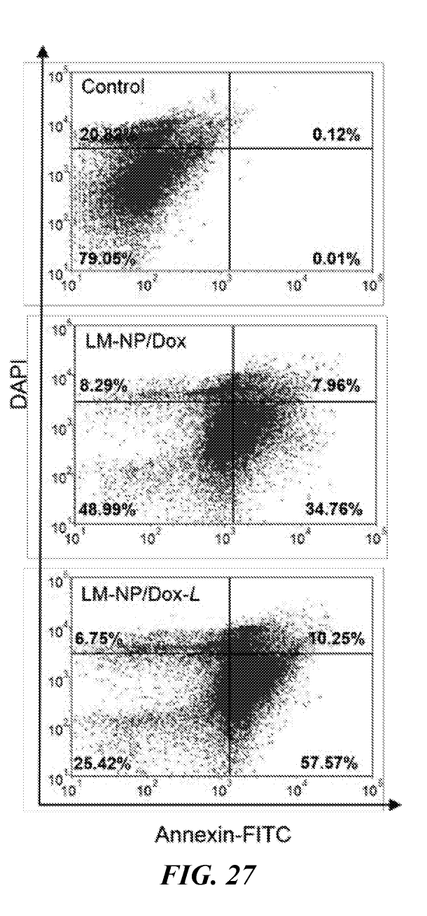

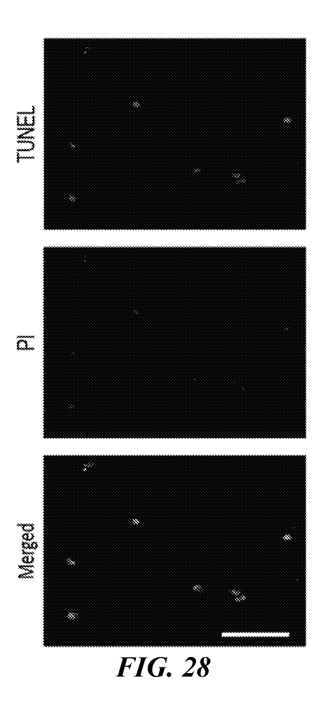

The total apoptotic ratio of HeLa cells incubated with doxorubicin loaded ligand-capped liquid metal nanoparticles was 68%, which was significantly higher than 43% of HeLa cells incubated with doxorubicin loaded liquid metal nanoparticles without the hyaluronic acid targeting ligand (LM-NP/Dox) (FIG. 27). In addition, the cells treated with doxorubicin loaded ligand-capped liquid metal nanoparticles showed extensive apoptotic DNA fragmentation stained by the Alexa Fluor 488 as green fluorescence (FIG. 28), substantiating that doxorubicin loaded ligand-capped liquid metal nanoparticles is an effective intracellular delivery vehicle for enhanced apoptosis-inducing activity

Next, the cytotoxicity of doxorubicin-loaded nanospheres towards HeLa cells was evaluated with 3-(4, 5-dimethylthiazol-2-yl)-2, 5-diphenyltetrazolium bromide (MTT) assay. HeLa cells (1×10⁴ cells per well) were seeded in the 96-well plates. After culture for 24 h, the cells were exposed to the doxorubicin solution and doxorubicin-loaded liquid metal nanoparticles with different concentrations of doxorubicin for 24 h, followed by adding 20 μl of the MTT solution (5 mg mL⁻¹). After 4 h of incubation, the medium was removed, and the cells were mixed with 150 μl of dimethyl sulfoxide. The absorbance was measured at a test wavelength of 570 nm and a reference wavelength of 630 nm by a microplate reader (Infinite M200 PRO, Tecan).

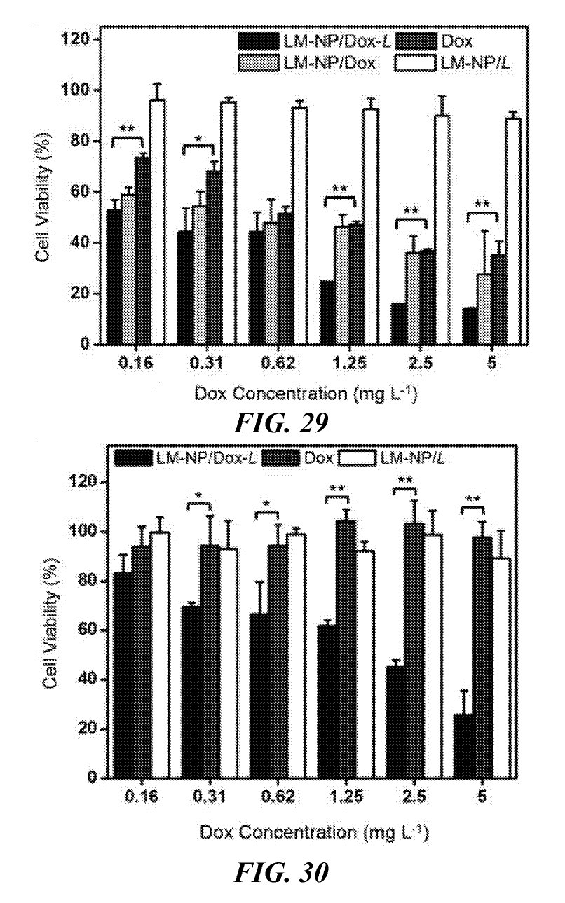

The results of the in vitro cytotoxicity assay indicated that cell viability was dependent on both doxorubicin concentration and incubation time (FIG. 29). The half-maximal inhibitory concentration (IC₅₀) of doxorubicin loaded liquid metal nanoparticles without the hyaluronic acid targeting ligand and doxorubicin loaded ligand-capped liquid metal nanoparticles towards HeLa cells for 24 h treatment were 0.81 mg L⁻¹ and 0.23 mg L⁻¹, respectively. The doxorubicin loaded ligand-capped liquid metal nanoparticles displayed higher cytotoxicity than the free doxorubicin solution (IC₅₀=1.33 mg L⁻¹), while the blank ligand-capped liquid metal nanoparticles without doxorubicin showed insignificant cytotoxicity within the range of tested concentrations. At low doxorubicin concentrations (0.16 and 0.31 mg L⁻¹), the doxorubicin loaded liquid metal nanoparticles without the hyaluronic acid targeting ligand also had higher cytotoxicity than the free doxorubicin solution. It was suggested that the acid-promoted release of doxorubicin achieved by liquid metal nanospheres provided higher cytotoxic activity towards cancer cells. The doxorubicin loaded ligand-capped liquid metal nanoparticles showed enhanced cytotoxicity (3.5-fold for 24 h treatment) compared with the doxorubicin loaded liquid metal nanoparticles without the hyaluronic acid targeting ligand toward HeLa cells that overexpress CD44 receptor (Zoller M et al. Nature Rev. Cancer 2011, 11, 254-267), which can be attributed to the hyaluronic acid targeting ligand.

Next, the in vitro cytotoxicity of doxorubicin loaded ligand-capped liquid metal nanoparticles towards doxorubicin-resistant HeLa cells was investigated. The results showed that the doxorubicin loaded ligand-capped liquid metal nanoparticles displayed significant cytotoxicity towards doxorubicin-resistant HeLa cells (IC₅₀=2.46 mg L⁻¹), while the free doxorubicin solution did not show any obvious cytotoxicity, even at a concentration of 5 mg L⁻¹ (FIG. 30). These results offer guidelines for future applications of liquid metal nanoparticles in the combination treatment of cancer drug resistance.

To evaluate the tumor targeting capability of doxorubicin loaded ligand-capped liquid metal nanoparticles, Cy5.5-labelled doxorubicin loaded ligand-capped liquid metal nanoparticles (Cy5.5-(LM-NP/Dox-L)) were administrated intravenously into the HeLa tumor-bearing mice. The animal study protocol was approved by the Institutional Animal Care and Use Committee at North Carolina State University and University of North Carolina at Chapel Hill. When the tumors reached to 200-400 mm³, the mice were intravenously injected by Cy5.5-labelled doxorubicin loaded liquid metal nanoparticles without the hyaluronic acid targeting ligand and Cy5.5-labelled doxorubicin loaded ligand-capped liquid metal nanoparticles at Cy5.5 dose of 30 nmol kg⁻¹. Cy5.5 labelled nanospheres were prepared by adding Cy5.5 conjugated HS-PEG-NH₂ (molecular weight: 1000) into the ethanol containing mixture. Cy5.5 is a near-infrared (IR) fluorescence-emitting cyanine dye (Excitation/emission maximum 678/694 nm). Images were taken on the IVIS Lumina imaging system (Caliper, USA) at 6, 24 and 48 h post injection.

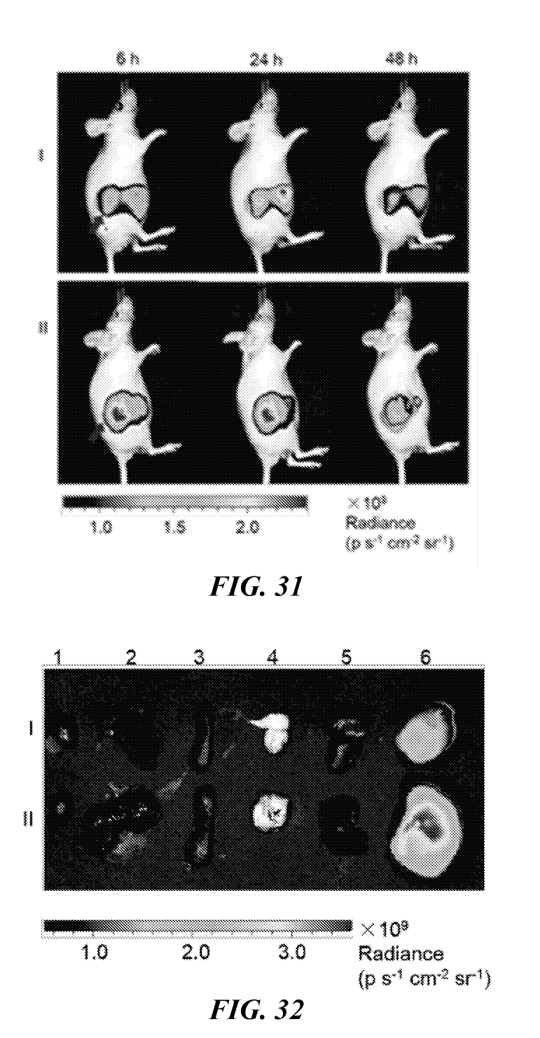

The results showed that within a short time period, the Cy5.5-labelled doxorubicin loaded ligand-capped liquid metal nanoparticles presented a stronger fluorescence signal in the tumor region compared with the Cy5.5-labelled doxorubicin loaded liquid metal nanoparticles without the hyaluronic acid targeting ligand (Cy5.5-(LM-NP/Dox)) (FIG. 31). As time increased, elevated fluorescence signals from the Cy5.5-labelled doxorubicin loaded ligand-capped liquid metal nanoparticles were observed at the tumor site as compared with the normal tissues within 48 h post injection, indicating a notable tumor targeting effect of the nanospheres.

After the 48 h scanning, the mice were euthanized. The tumors as well as maj or organs were harvested, weighed and subjected for ex vivo imaging. Region-of-interests were circled around the organs, and the fluorescence intensities were analyzed by Living Image Software.

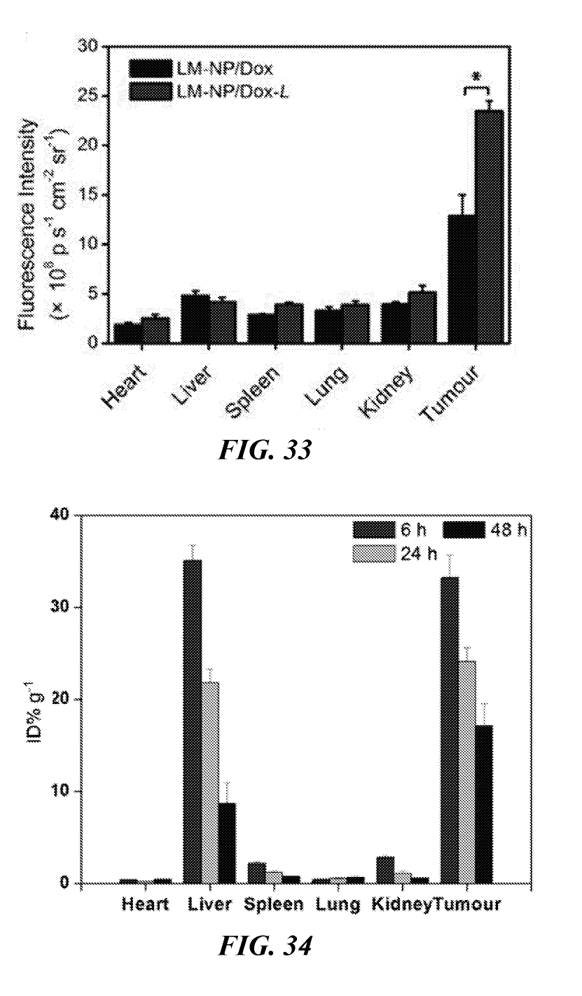

The intensity of fluorescence signal of the Cy5.5-labelled doxorubicin loaded ligand-capped liquid metal nanoparticles at the tumor site was significantly higher than that of the Cy5.5-labeled doxorubicin loaded liquid metal nanoparticles without the hyaluronic acid targeting ligand (FIG. 32). Based on the quantitative region-of-interest analysis, the fluorescence intensity of the Cy5.5-labelled doxorubicin loaded ligand-capped liquid metal nanoparticles at the tumor site was 1.82-fold that of the Cy5.5-labelled doxorubicin loaded liquid metal nanoparticles without the hyaluronic acid targeting ligand (FIG. 33). In addition, the fluorescence signal at the tumor site was three times higher than that in the liver or kidney (FIG. 32 and FIG. 33). The potent tumor targeting capability of the doxorubicin loaded ligand-capped liquid metal nanoparticles can be attributed to the combination of an enhanced permeability and retention (EPR) effect and active targeting mechanisms.

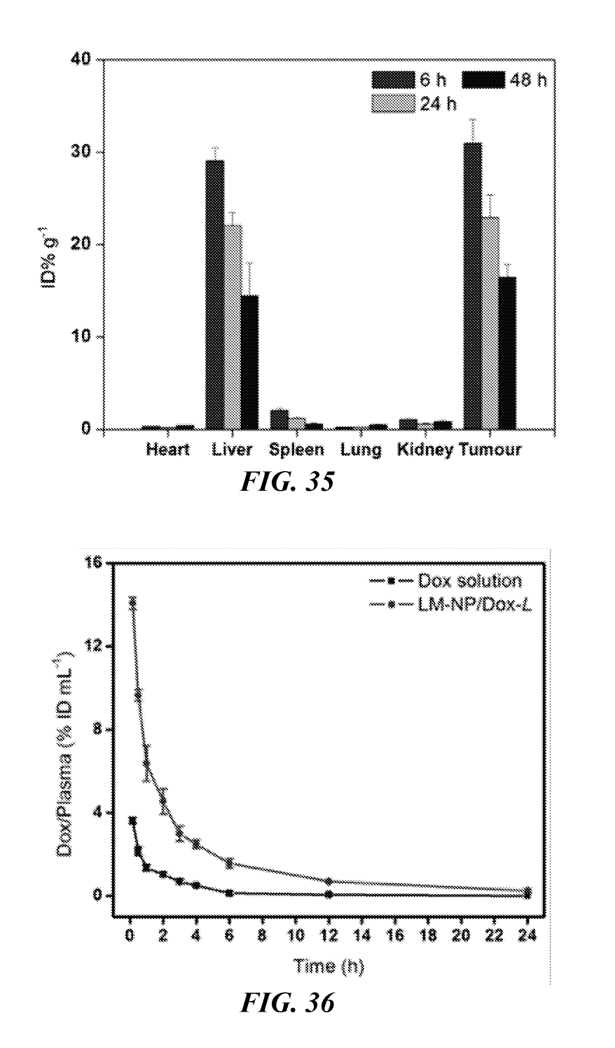

The time-dependent biodistribution of ligand-capped liquid metal nanoparticles was further quantified by ICP-MS. HeLa tumor-bearing nude mice were sacrificed at 6 h, 24 h and 48 h after intravenous injection of doxorubicin loaded ligand-capped liquid metal nanoparticles for tissue collection. The results further substantiated the potent tumor targeting capability of doxorubicin loaded ligand-capped liquid metal nanoparticles (FIG. 34 and FIG. 35). The pharmacokinetics of doxorubicin loaded ligand-capped liquid metal nanoparticles administered intravenously into mice was evaluated by quantitatively monitoring the doxorubicin concentration in blood plasma (FIG. 36). The elimination half-life (t_{1/2}), the mean residence time and the area under the curve depicting the plasma drug concentration versus time were significantly higher than those of the free doxorubicin solution, suggesting the capability of doxorubicin loaded ligand-capped liquid metal nanoparticles to maintain a high drug concentration during a prolonged systemic circulation (Table 2).

TABLE 2
Pharmacokinetic parameters of the doxorubicin
solution and doxorubicin loaded ligand-capped
liquid metal nanoparticles (LM-NP/Dox-L).

| | | Doxorubicin loaded ligand- |
|---|---|---|
| | Doxorubicin | capped liquid metal nanoparticles |
| Parameter | (Dox) | (LM-NP/Dox-L) |
| Cₘₐₓ^{a} (μg mL⁻¹) | 1.45 ± 0.06 | 5.63 ± 0.12** |
| AUC^{b} (μg (mL × h)⁻¹) | 2.75 ± 0.12 | 16.55 ± 0.94** |
| t_{1/2}^{c} (h) | 1.11 ± 0.02 | 6.30 ± 1.22** |
| MRT^{d} (h) | 2.80 ± 0.61 | 6.38 ± 0.68** |

^{a}Maximum doxorubicin concentration in plasma
^{b}Area under the plasma doxorubicin versus time curves
^{c}Elimination half-life
^{d}Mean residence time
Data are means ± standard deviation (n = 3)
**P < 0.01 compared with the doxorubicin solution (two-tailed Student's t-test)

The antitumor efficacy of doxorubicin loaded ligand-capped liquid metal nanoparticles was further assessed in the HeLa tumor-bearing xenograft mice. The tumor-bearing mice were weighed and randomly divided into different groups when the tumor volume reached to 50 mm³. From Day 0, the mice were intravenously injected with doxorubicin solution (2 mg kg⁻¹), doxorubicin loaded liquid metal nanoparticles without the hyaluronic acid targeting ligand (2 mg kg⁻¹), doxorubicin loaded ligand-capped liquid metal nanoparticles (2 mg kg⁻¹), and saline as a negative control every other day for 12 days, and meanwhile the tumor size was measured. At Day 14, the mice were euthanized, and the tumor as well as the heart were collected, weighed, washed with saline thrice, and fixed in 10% neutral-buffered formalin.

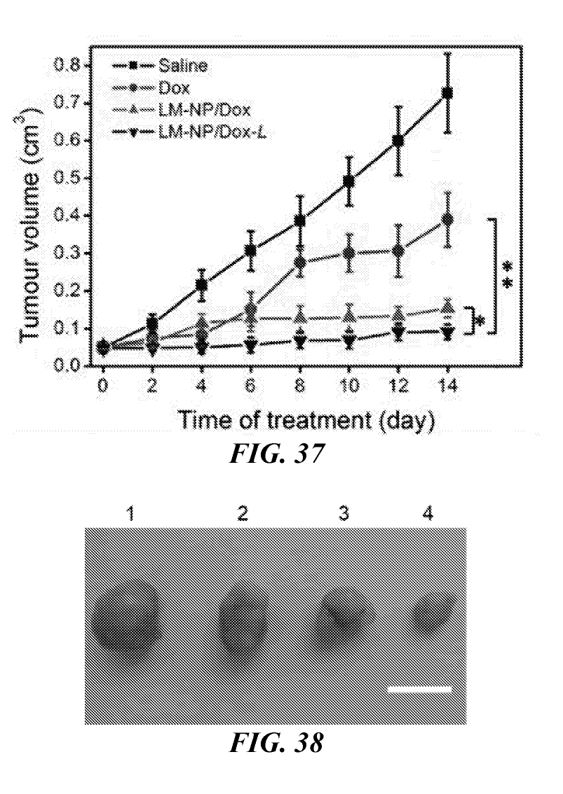

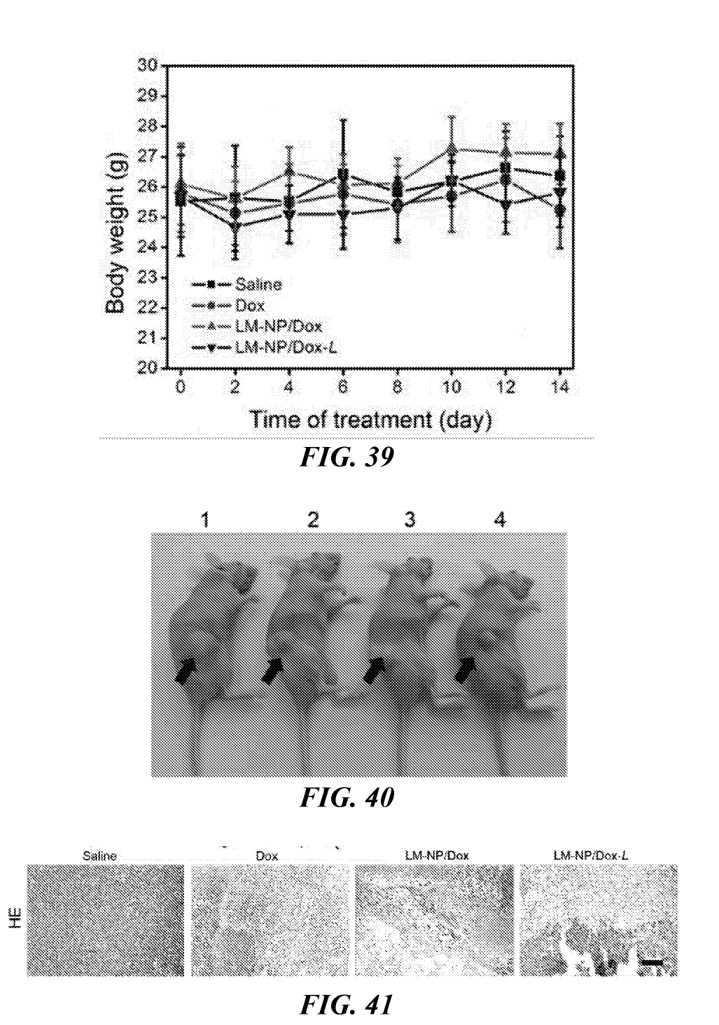

Different doxorubicin formulations displayed significant tumor inhibition effects compared with saline as a negative control after successive intravenous administration into the HeLa tumor-bearing mice (FIG. 37). Both the doxorubicin loaded liquid metal nanoparticles without the hyaluronic acid targeting ligand and the doxorubicin loaded ligand-capped liquid metal nanoparticles displayed higher inhibition efficacy toward HeLa tumor growth than the free doxorubicin solution, which can result from the tumor targeting capability of these nanospheres. A difference in tumor-volume inhibition between the doxorubicin loaded ligand-capped liquid metal nanoparticles and the doxorubicin loaded liquid metal nanoparticles without the hyaluronic acid targeting ligand was also observed (FIG. 37 and FIG. 38), further demonstrating the enhanced active targeting capability of the doxorubicin loaded ligand-capped liquid metal nanoparticles. No significant change of mice body weights was observed during the treatment with the doxorubicin loaded ligand-capped liquid metal nanoparticles (FIG. 39 and FIG. 40).

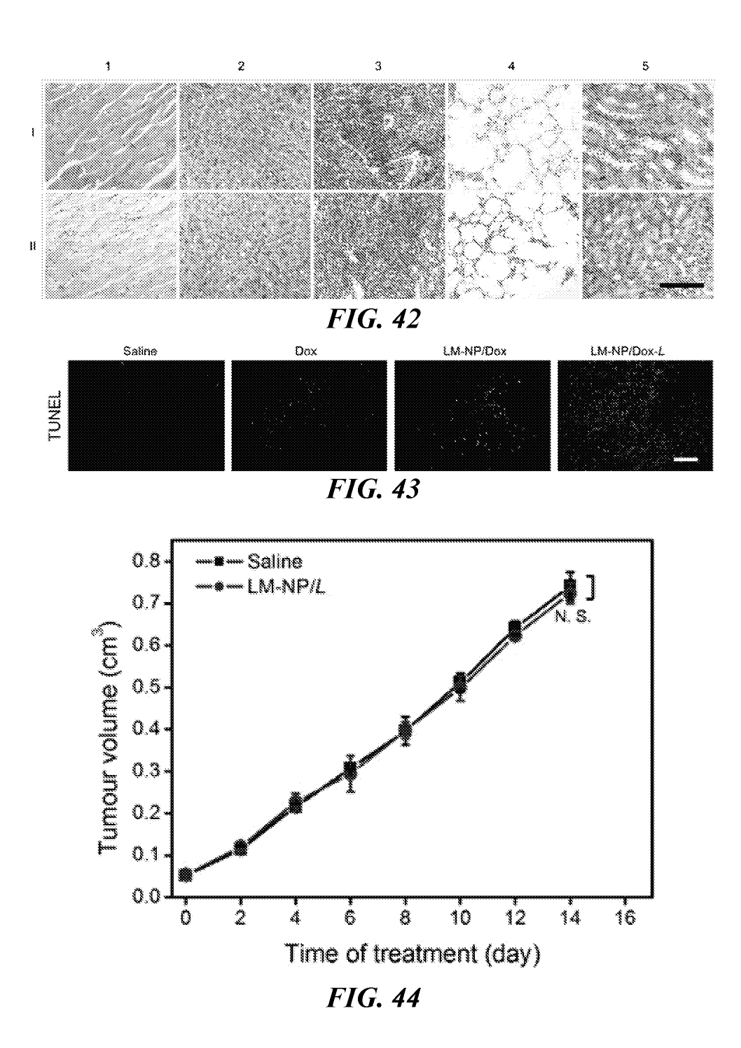

The histologic images using hematoxylin and eosin (H&E) staining showed that cancer cell remission occurred in the tumor tissue (FIG. 41) after doxorubicin loaded ligand-capped liquid metal nanoparticles administration, with no obvious pathological abnormalities in normal organs, including cardiomyopathy, the major toxic effect of doxorubicin cancer treatment (FIG. 42) (Takemura G et al. Prog. in Cardiovasc. Dis. 2007, 49, 330-352). For the hematoxylin and eosin staining, the formalin-fixed tumors and hearts were embedded in paraffin blocks and visualized by optical microscope (DM5500B, Leica).

Moreover, the images obtained using an in situ TUNEL assay showed the highest level of cell apoptosis in the tumor harvested from the mice treated with the doxorubicin loaded ligand-capped liquid metal nanoparticles (FIG. 43). These results indicate the enhanced tumor inhibition activity of the doxorubicin loaded ligand-capped liquid metal nanoparticles can be attributable in part to increased apoptosis induced by the doxorubicin loaded ligand-capped liquid metal nanoparticles. For the TUNEL apoptosis staining, the fixed tumor sections were stained by the In Situ Cell Death Detection Kit (Roche Applied Science) according to the manufacturer's protocol. Hoechst 33342 was used for nuclear counterstaining. The stained tumor slides were observed by fluorescence microscope (IX71, Olympus).

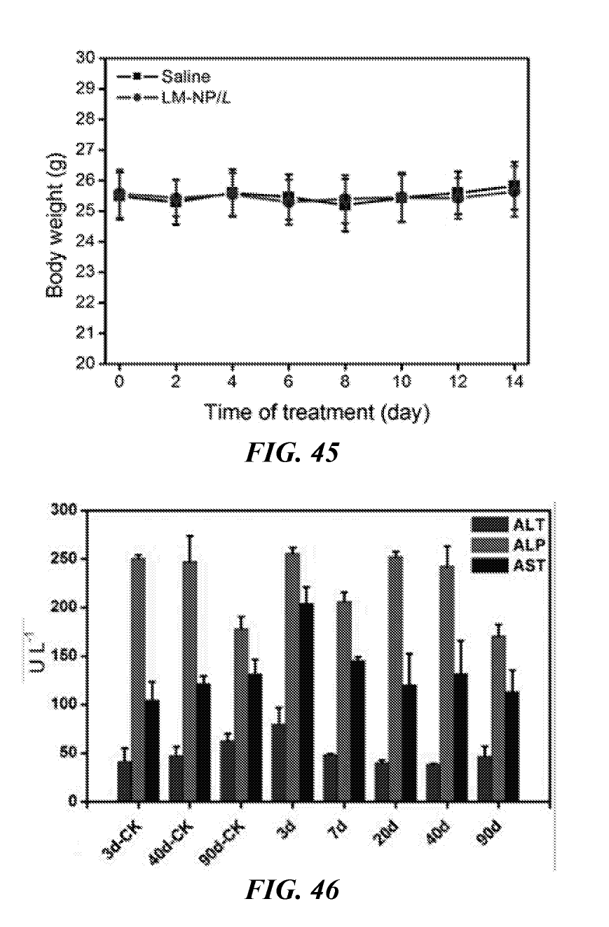

As a control, the anticancer behavior of blank ligand-capped liquid metal nanoparticles (e.g., without any drug loading) was also explored in tumor-bearing mice. No significant anticancer effects were observed after intravenous injection of the blank ligand-capped liquid metal nanoparticles (FIG. 44). No significant change of mice body weights was observed during the treatment with the blank ligand-capped liquid metal nanoparticles (FIG. 45). Collectively, these results indicated that the hyaluronic acid conjugated doxorubicin loaded ligand-capped liquid metal nanoparticles efficiently accumulated at the tumor site, indicating effective receptor-mediated intracellular transport, and thereby enhanced antitumor efficacy in vivo.

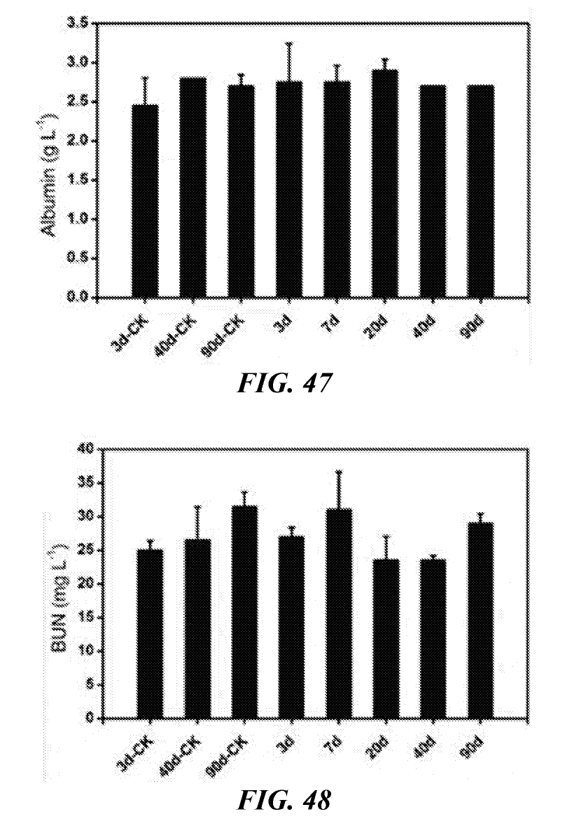

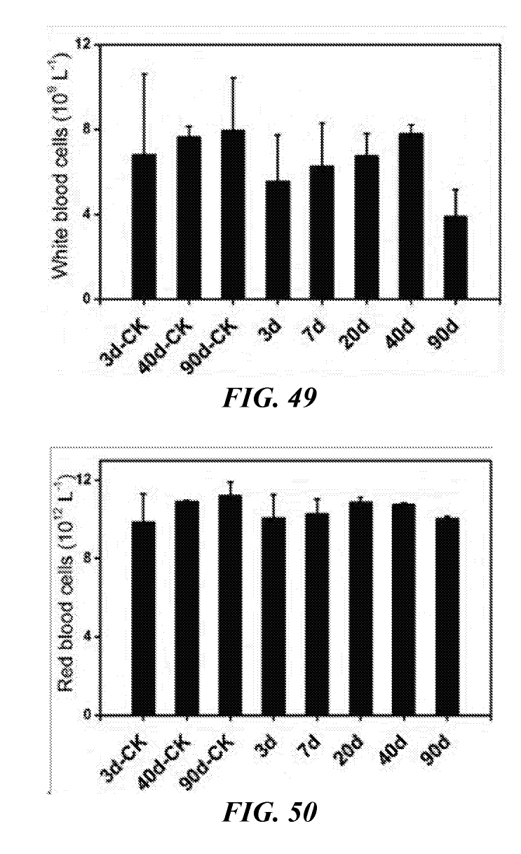

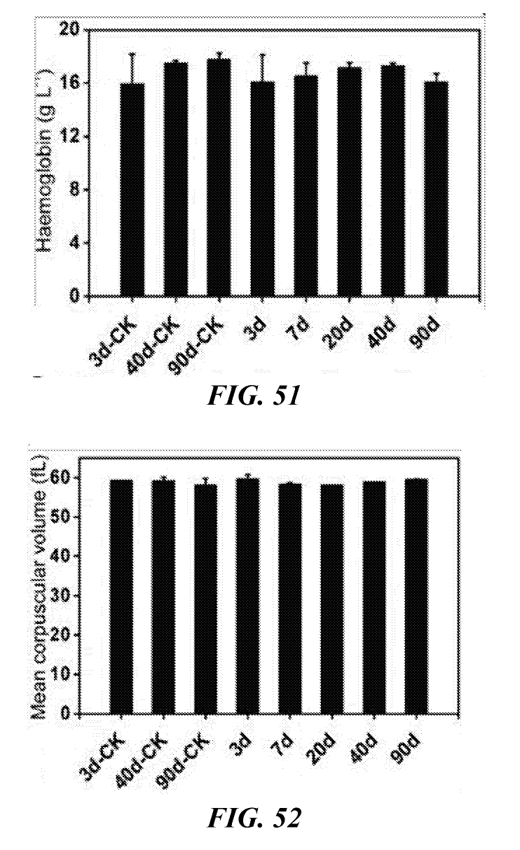

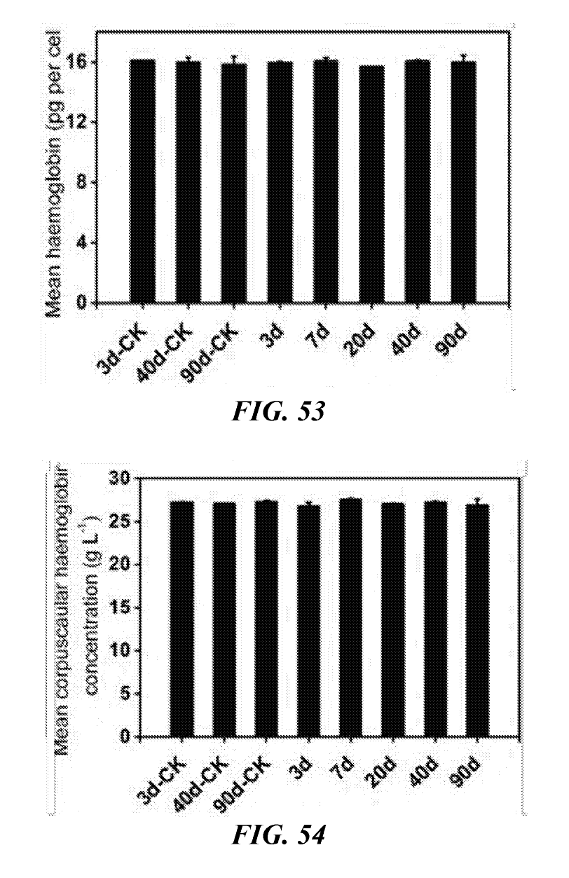

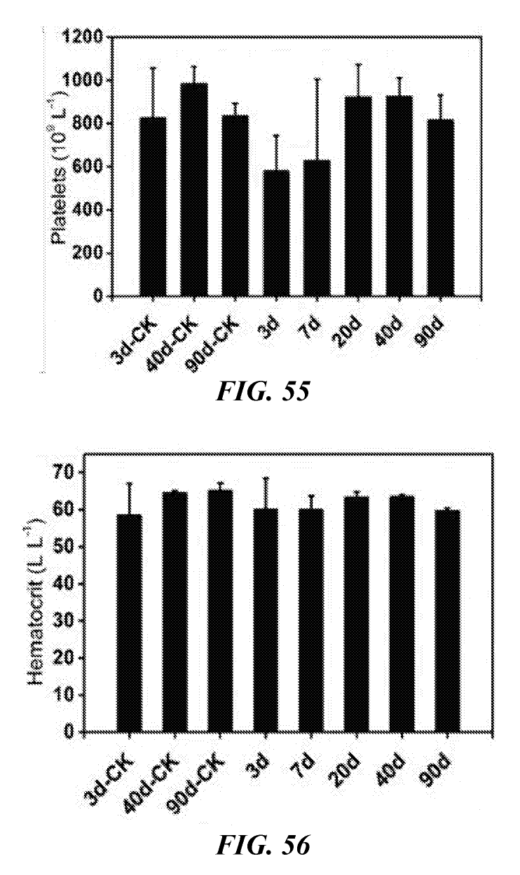

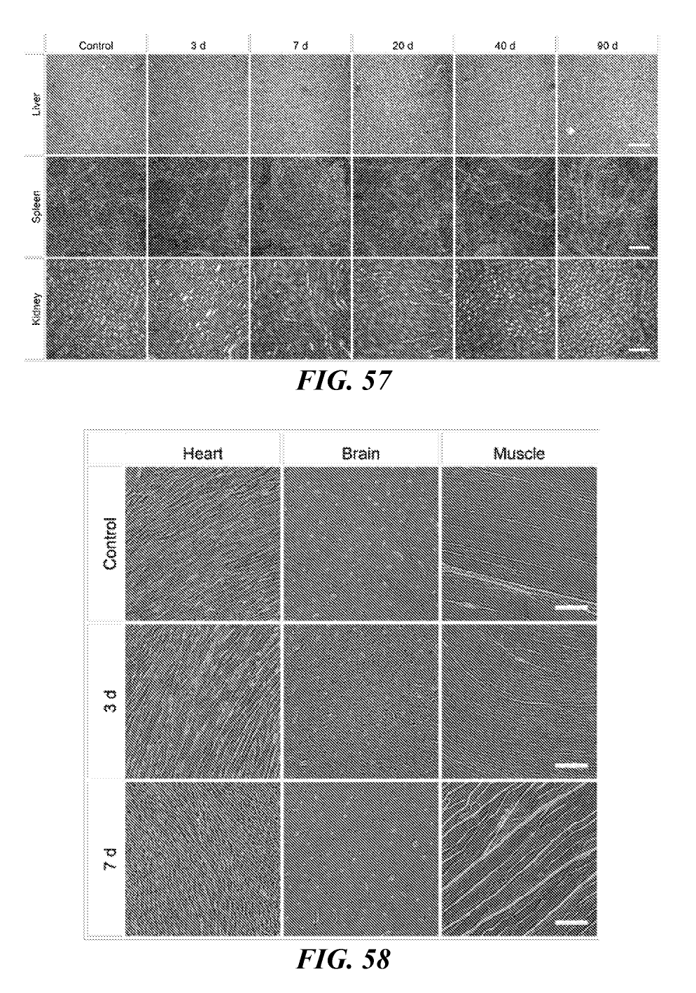

The toxicology of the ligand-capped liquid metal nanoparticles to female Balb/c mice was investigated over 3 months. The female Balb/c mice injected with ligand-capped liquid metal nanoparticles (45 mg kg⁻¹) were sacrificed at 3, 7, 20, 40 and 90 days post injection for blood collection. The levels of liver function markers (e.g., alanine aminotransferase (ALT), aspartate aminotransferase (AST), alkaline phosphatase (ALP)) (FIG. 46) and the albumin concentration (FIG. 47) were all within reference ranges (Ynag K et al. ACS Nano 2011, 5, 516-522; Zaias J et al. J. Am. Assoc. Lab. Anim. Sci. 2009, 48, 387), indicating that no obvious hepatic toxicity was induced by the ligand-capped liquid metal nanoparticle treatment. As an indicator of the kidney functions, the urea levels (BUN) in the blood of treated mice were examine, and were also found to be within the normal range (FIG. 48). For hematological assessment, white blood cells (FIG. 49), red blood cells (FIG. 50), hemoglobin (FIG. 51), mean corpuscular volume (FIG. 52), mean corpuscular hemoglobin (FIG. 53), mean corpuscular hemoglobin concentration (FIG. 54), platelet count (FIG. 55), and hematocrit (FIG. 56) were examined. Although a variation of platelets concentration was observed on day 3, the value itself still fell into the reference normal range (Ynag K et al. ACS Nano 2011, 5, 516-522) (FIG. 55). In addition, a steady recovery in the platelets level was observed over time (FIG. 55). The platelets level fully recovered on day 20, with no significant difference compared to that of the age control group (FIG. 55). All of the above indices in the ligand-capped liquid metal nanoparticle treated groups were normal compared with the control groups (FIGS. 48-56). No noticeable organ damage was observed during necropsy of the sacrificed mice (FIG. 57). Tissues collected from heart, brain and muscle were also investigated, and no obvious tissue injury was observed (FIG. 58). Taken together, the ligand-capped liquid metal nanoparticles displayed no obvious toxicity at the treatment dose, which can be important for the application of ligand-capped liquid metal nanoparticles as nanomedicine.

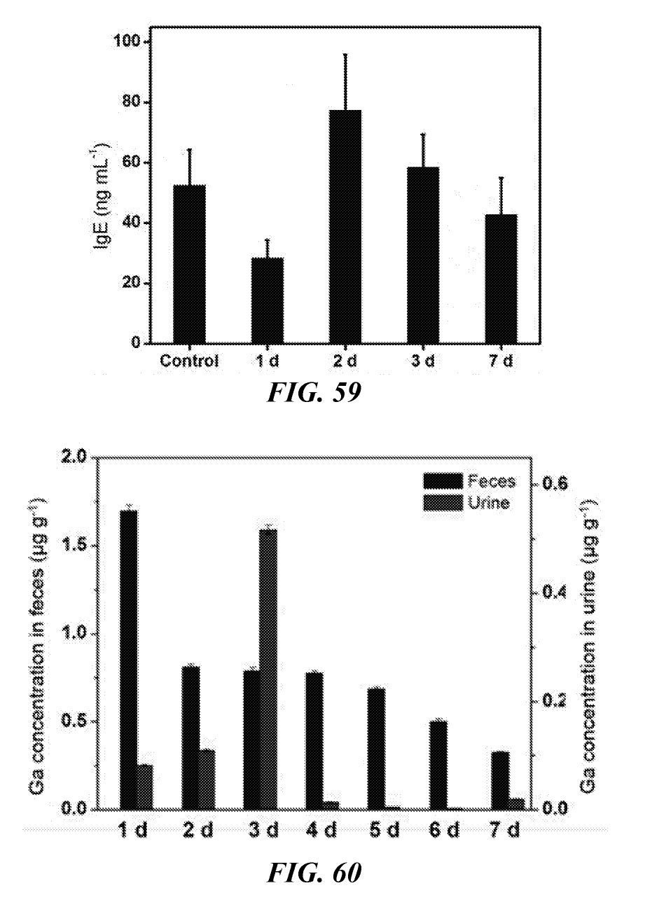

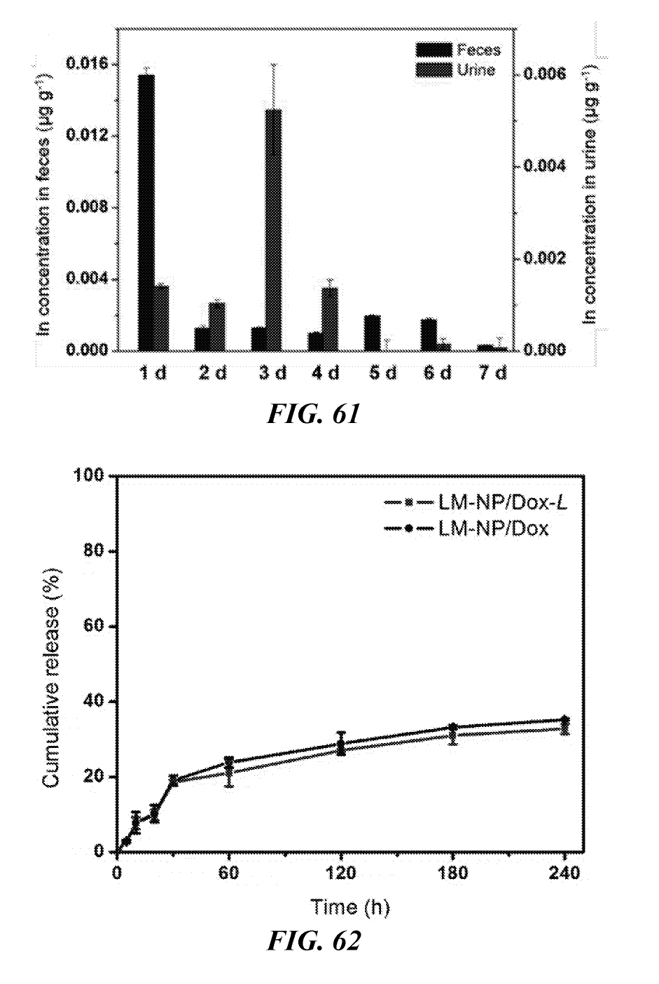

The single-dose, acute toxicity of ligand-capped liquid metal nanoparticles and doxorubicin loaded ligand-capped liquid metal nanoparticles to female Balb/c mice was investigated by identifying the maximum tolerated dose (MTD). The maximum tolerated dose (MTD) was defined as the highest dose that does not cause major adverse reactions in mice over 10 days post single dose intravenous injection (Yu T et al. ACS Nano 2012, 6, 2289-2301). In this study, major adverse reactions were considered to be immediate death, impaired mobility or irregular breathing that could not be recovered within a day, over 10% weight loss over continuous days, or histological evidence of organ toxicity. The estimated maximum tolerated dose of the ligand-capped liquid metal nanoparticles was determined to be 700 mg kg⁻¹, suggesting low acute toxicity. The estimated maximum tolerated dose of doxorubicin loaded ligand-capped liquid metal nanoparticles was found to be 55 mg kg⁻¹ (doxorubicin dose), 1.75-fold higher than that of free doxorubicin solution. In addition, no obvious increase in IgE production was observed in ligand-capped liquid metal nanoparticle injected mice, revealing no sign of allergic reactions (FIG. 59). Time-dependent excretion was also monitored to study the in vivo metabolism of ligand-capped liquid metal nanoparticles. The results suggested that the clearance of ligand-capped liquid metal nanoparticles was possibly through both fecal and renal excretions (FIG. 60 and FIG. 61). The excreted concentrations of both gallium and indium steadily decreased over time.

A liquid metal-based drug delivery platform for anticancer therapy was discussed herein. The formulation was formed and tailored via ligand-mediated self-assembly using sonication. The resulting liquid-metal nanospheres loaded with doxorubicin (Dox) had an average diameter of 107 nm and were able to fuse for promoting drug release and eventually degrade under a mild acidic environment, which can facilitate release of doxorubicin in acidic endosomes after cellular internalization. After fusing, these nanospheres also displayed a contrast enhancing capability when imaged by X-ray, suggesting potential as a theranostic reagent. Equipped with hyaluronic acid, a tumor-targeting ligand, this formulation displayed enhanced chemotherapeutic inhibition toward the xenograft tumor-bearing mice. Systematic investigation of toxicology of the ligand-capped liquid metal nanoparticles revealed no obvious toxicity at the treatment dose, favoring biomedical applications. This metal-based drug delivery system with bioconjugation flexibility and fusible and degradable behavior under physiological conditions can provide a strategy for engineering theranostic agents with low toxicity.

Moreover, considering other physical properties of liquid metal, including capability of “dissolving” iron or other metals, mechanical flexibility toward tissues and electrical conductivity, many new materials and scaffolds integrated with liquid metal for drug delivery and tissue engineering can be envisioned.

Other advantages which are obvious and which are inherent to the invention will be evident to one skilled in the art. It will be understood that certain features and sub-combinations are of utility and may be employed without reference to other features and sub-combinations. This is contemplated by and is within the scope of the claims. Since many possible embodiments may be made of the invention without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. A biocompatible particle comprising: a metallic core, a plurality of drug-loading ligands coordinated to the metallic core, and a plurality of targeting ligands coordinated to the metallic core, wherein the metallic core has a melting point of 100° C. or less, and wherein the biocompatible particle is pH sensitive.

2. (canceled)

3. The biocompatible particle of claim 1, wherein the metallic core is liquid at physiological temperatures.

4. The biocompatible particle of claim 1, wherein the metallic core comprises gallium, indium, tin, zinc, or a combination thereof.

5. (canceled)

6. The biocompatible particle of claim 1, wherein the metallic core comprises a eutectic gallium-indium alloy.

7. The biocompatible particle of claim 1, wherein the metallic core comprises from 95% to 75% gallium and from 25% to 5% indium by weight.

8. The biocompatible particle of claim 1, wherein the plurality of drug-loading ligands comprise natural or synthetic small molecules, macromolecules, nanostructures, drug derivatives, or combinations thereof.

9. The biocompatible particle of claim 1, wherein the plurality of drug-loading ligands comprise cyclodextrin.

10. (canceled)

11. (canceled)

12. (canceled)

13. The biocompatible particle of claim 1, wherein the plurality of targeting ligands comprise a plurality of tumor-targeting ligands.

14. (canceled)

15. The biocompatible particle of claim 1, wherein the plurality of targeting ligands comprise hyaluronic acid.

16. (canceled)

17. The biocompatible particle of claim 1, further comprising a therapeutically effective amount of a drug coordinated to the plurality of drug-loading ligands, wherein the drug comprises a chemotherapy drug, anti-inflammatory drug, anti-infective drug, or regenerative drug.

18. (canceled)

19. The biocompatible particle of claim 17, wherein the drug comprises doxorubicin, paclitaxel, or cisplatin.

20. The biocompatible particle of claim 1, wherein the average particle size of the biocompatible particle is from 5 nm to 2 μm.

21. The biocompatible particle of claim 20, wherein the average particle size of the biocompatible particle is 200 nm or less.

22. (canceled)

23. A pH-triggered X-ray contrast comprising the biocompatible particle of claim 1.

24. A pH-triggered drug-delivery particle comprising the biocompatible particle of claim 1 with a therapeutically effective amount of a drug coordinated to the plurality of drug-loading ligands.

25. A pH-triggered biodegradable particle comprising the biocompatible particle of claim 1.

26. (canceled)

27. A method of making the biocompatible particle of claim 1, the method comprising mixing a mixture comprising a metal having a melting point of 100° C. or less, a plurality of drug-loading ligands, and a plurality of targeting ligands, thereby forming the biocompatible particle.

28-48. (canceled)

49. A method of treating cancer in a subject comprising administering to the subject a therapeutically effective amount of the biocompatible particle of claim 1.

50. (canceled)

51. A method of suppressing tumor growth in a subject, comprising contacting at least a portion of the tumor with a therapeutically effective amount of the biocompatible particle of claim 1.

52. A method of imaging a cell or a population of cells within or about a subject, the method comprising administering to the subject an amount of the biocompatible particle of claim 1; and detecting the biocompatible particle of claim 1.

53. (canceled)
